SEQUENCE LISTING (28 sequences)

SEQ ID NO: 1 - DNA, 34 bp, Artificial Sequence
  Primer
tggactagggtctcgcgggaatagtgggtactgc

SEQ ID NO: 2 - DNA, 41 bp, Artificial Sequence
  Primer
cgctcctaggtctcattataacaaaagcatgggcagttacg

SEQ ID NO: 3 - DNA, 36 bp, Artificial Sequence
  Primer
tggactagggtctctatcctagaaatcgctgtcgcc

SEQ ID NO: 4 - DNA, 36 bp, Artificial Sequence
  Primer
cgctcctaggtctcataggcatgtgattggtgggtc

SEQ ID NO: 5 - DNA, 18 bp, Artificial Sequence
  Primer
ggcggtagaagtcttagt

SEQ ID NO: 6 - DNA, 19 bp, Artificial Sequence
  Primer
ggagaaggagaaatgatgg

SEQ ID NO: 7 - DNA, 20 bp, Artificial Sequence
  Primer
ggccaccacactcctattgt

SEQ ID NO: 8 - DNA, 20 bp, Artificial Sequence
  Primer
atgctgcggcttcaaatccg

SEQ ID NO: 9 - DNA, 34 bp, Artificial Sequence
  Primer
cccccatgcctcaggatactcctcaatagtgatc

SEQ ID NO: 10 - DNA, 28 bp, Artificial Sequence
  Primer
tgattgttagcggtgtggtcgggtgtgt

SEQ ID NO: 11 - DNA, 30 bp, Artificial Sequence
  Primer
ccacttcttaccacaaggcacacctacacc

SEQ ID NO: 12 - DNA, 28 bp, Artificial Sequence
  Primer
aggcctagtatgaggagcgttatggagt

SEQ ID NO: 13 - DNA, 18 bp, Artificial Sequence
  Primer
ggcggtagaagtcttagt

SEQ ID NO: 14 - DNA, 19 bp, Artificial Sequence
  Primer
ggagaaggagaaatgatgg

SEQ ID NO: 15 - DNA, 21 bp, Artificial Sequence
  Primer′
gagcgggaatagtgggtactg

SEQ ID NO: 16 - DNA, 21 bp, Artificial Sequence
  Primer
acaaaagcatgggcagttacg

SEQ ID NO: 17 - DNA, 20 bp, Artificial Sequence
  Primer
ggccaccacactcctattgt

SEQ ID NO: 18 - DNA, 20 bp, Artificial Sequence
  Primer
atgctgcggcttcaaatccg

SEQ ID NO: 19 - DNA, 25 bp, Artificial Sequence
  Primer
caagccctactaattaccattatac

SEQ ID NO: 20 - DNA, 21 bp, Artificial Sequence
  Primer
agtccatggaatccagtagcc

SEQ ID NO: 21 - DNA, 20 bp, Artificial Sequence
  Primer
cccccgagcaatctcaatta

SEQ ID NO: 22 - DNA, 28 bp, Artificial Sequence
  Primer
tgattgttagcggtgtggtcgggtgtgt

SEQ ID NO: 23 - DNA, 29 bp, Artificial Sequence
  Primer
cccccatgcctcaggatactcctcaatag

SEQ ID NO: 24 - DNA, 28 bp, Artificial Sequence
  Primer
tgattgttagcggtgtggtcgggtgtgt

SEQ ID NO: 25 - DNA, 30 bp, Artificial Sequence; identical to SEQ ID NO: 11
  Primer

SEQ ID NO: 26 - DNA, 28 bp, Artificial Sequence
  Primer
aggcctagtatgaggagcgttatggagt

SEQ ID NO: 27 - DNA, 20 bp, Artificial Sequence
  Primer
ctgactatcctagaaatcgc

SEQ ID NO: 28 - DNA, 20 bp, Artificial Sequence
  Primer
gattggtgggtcattatgtg

(19) United States
(12) Patent Application Publication
(10) Pub. No.: US 2016/0304854 A1
(43) Pub. Date: Oct. 20, 2016

(54) MITOCHONDRIAL GENOME EDITING

(71) Applicant: GenOva Laboratories LLC, New York, NY (US)

(72) Inventors: Juan Carlos Izpisua Belmonte, La Jolla, CA (US); Pradeep Reddy Dubbaka Venu, San Diego, CA (US); Alejandro Ocampo, San Diego, CA (US); Keiichiro Suzuki, San Diego, CA (US); Yuji Tsunekawa, Chuo-ku Kobe-shi (JP)

(73) Assignee: GenOva Laboratories LLC, New York, NY (US)

(21) Appl. No.: 15/130,891

(22) Filed: Apr. 15, 2016

Related U.S. Application Data
(60) Provisional application No. 62/151,991, filed on Apr. 23, 2015.
(60) Provisional application No. 62/148,723, filed on Apr. 16, 2015.

(51) Int. Cl. C12N 15/10; A61B 17/435; C12N 5/071

(52) U.S. Cl. CPC C12N 15/102; C12N 5/0612; A61B 17/435

(57) ABSTRACT

Methods of preventing the transmission of a mitochondrial disease, disorder, or condition using mitochondria-targeted enzymes or mRNA encoding mitochondria-targeted enzymes. The methods as described herein can specifically eliminate mitochondrial DNA (mtDNA) mutations in the germline.

### STATEMENT REGARDING FEDERALLY SPONSORED RESEARCH OR DEVELOPMENT

Not Applicable.

### MATERIAL INCORPORATED-BY-REFERENCE

The Sequence Listing, which is a part of the present disclosure, includes a computer readable form comprising nucleotide or amino acid sequences of the present invention. The subject matter of the Sequence Listing is incorporated herein by reference in its entirety.

### BACKGROUND OF THE INVENTION

Diseases resulting from mitochondrial dysfunction caused by mitochondrial DNA (mtDNA) mutations affect 1 in 5,000 children (Haas et al., 2007), and it is estimated that 1 in 200 women could be a mitochondrial disease carrier. Currently, there is no cure for mitochondrial diseases. Genetic counseling and pre-implantation genetic diagnosis (PGD) represent the only therapeutic options for preventing transmission of mitochondrial diseases caused by mtDNA mutations. However, due to the non-Mendelian segregation of mtDNA, PGD can only partially reduce the risk of transmitting the disease (Brown et al., 2006). Moreover, analysis of multiple blastomeres may compromise embryo viability. Recently, mitochondrial replacement techniques by spindle, pronuclear, or polar body genome transfer into healthy enucleated donor oocytes or embryos have been reported (Craven et al., 2010; Paull et al., 2013; Tachibana et al., 2013; Wang et al., 2014). Application of these techniques implies combining genetic material from three different individuals, which has raised ethical, safety, and medical concerns (Hayden, 2013; Vogel, 2014).

The relative levels of mutated and wildtype mtDNA can be altered in patient somatic cells containing the m.8993T>G mtDNA mutation responsible for the NARP and MILS syndromes, where elimination of mutated mtDNA led to the restoration of normal mitochondrial function (Alexeyev et al., 2008). Similarly, using the heteroplasmic NZB/BALB mouse model that carries two different mtDNA haplotypes (NZB and BALB), BALB mtDNA, which contains a unique ApaLI site, can be specifically reduced in vivo using a mitochondria-targeted ApaLI (Bacman et al., 2012; 2010). Transcription activator-like effector nucleases (TALENs) and zinc finger nucleases (ZFNs) targeted to mitochondria can be utilized for the specific elimination of mitochondrial genomes carrying mutations responsible for mitochondrial diseases (Bacman et al., 2013; Gammage et al., 2014; Minczuk et al., 2006; 2008). These approaches can allow for the targeting of a wider spectrum of mutations against which restriction endonucleases could not be used.

### SUMMARY OF THE INVENTION

Among the various aspects of the present disclosure is the provision of a method of preventing the transmission of a mitochondrial disease, disorder, or condition using mitochondria-targeted enzymes or mRNA encoding mitochondria-targeted enzymes to specifically eliminate mutated mitochondrial DNA (mtDNA) in the germline.

### DESCRIPTION OF THE DRAWINGS

The drawings, described below, are for illustrative purposes only and are not intended to limit the scope of the present teachings in any way. The patent or application file contains at least one drawing executed in color. Copies of this patent or patent application publication with color drawing(s) will be provided by the Office upon request and payment of the necessary fee.

FIG. 1 is a graphical image showing mitochondria-targeted nucleases selectively reduce mitochondrial DNA (mtDNA) haplotypes in germline. Germline heteroplasmy shift prevents transmission of mtDNA haplotypes to offspring. Human mutated mtDNA reduce in oocytes by mitochondria-targeted nucleases.

FIG. 2A-2E are a series of graphical images, microscopy images, and graphs showing heteroplasmy shift in NZB/BALB MII oocytes using mito-ApaLI. FIG. 2A is an illustration showing the injection of mito-ApaLI mRNA in oocytes for induction of heteroplasmy shift. FIG. 2B is a series of immunofluorescence images showing mitochondrial co-localization of mito-GFP and mito-ApaLI with Mitotracker in injected oocytes by immunofluorescence. Scale bars, 10 μm. FIG. 2C is an image of a representative gel from the RFLP analysis and a bar graph showing the quantification of mtDNA heteroplasmy in control and mito-ApaLI injected MII oocytes after 48 hr (Control n=16; mito-ApaLI n=12). FIG. 2D and FIG. 2E are a respective bar graph and whisker plot showing quantification of mtDNA copy number by qPCR in control and mito-ApaLI-injected oocytes MII after 48 hr (Control n=12; mito-ApaLI n=12). Error bars represent±SEM. ****p<0.0001.

FIG. 3A-3H are a series of images and graphs showing induction of heteroplasmy shift in NZB/BALB tail tip fibroblasts and oocytes, related to FIG. 2. FIG. 3A is a series of immunofluorescence images showing mitochondrial co-localization of mito-ApaLI with Mitotracker. Scale bars, 10 μm. FIG. 3B is an image of a representative gel from a RFLP analysis of mtDNA heteroplasmy in NZB/BALB cells transfected with mito-GFP, ApaLI and mito-ApaLI after 72 hr. FIG. 3C is a bar graph of the quantification of mtDNA levels by qPCR in NZB/BALB cells transfected with mito-GFP, ApaLI and mito-ApaLI after 72 hr. FIG. 3D is a series of images of representative gels from RFLP analysis and a bar graph showing the quantification of mtDNA heteroplasmy in control and mito-ApaLI-injected oocytes after 48 hr at polymorphic HindIII site (Control n=8; mito-ApaLI n=7). FIG. 3E and FIG. 3G and FIG. 3H are each respectively a bar graph and a whisker plot showing the quantification of mtDNA copy number by qPCR in control and mito-ApaLI-injected oocytes after 48 hr at polymorphic HindIII site (Control n=4; mito-ApaLI n=8). FIG. 3F is a whisker plot showing the analysis of mito-ApaLI specificity in BALB and NZB single haplotype oocytes.

Quantification of mtDNA copy number by qPCR in BALB (Control n=14; mito-ApaLI n=12) and NZB (Control n=8; mito-ApaLI n=5) MII oocytes 48 hr after injection of mito-ApaLI mRNA. Error bars represent±SEM. **p<0.01. ****p=0.0001. ns, non-significant.

FIG. 4A-4E are a series of images and graphs showing heteroplasmy shift in NZB/BALB embryos using mito-ApaLI. FIG. 4A is an illustration showing the injection of mito-ApaLI mRNA in one-cell embryos for induction of heteroplasmy shift. FIG. 4B is a series of images showing in vitro development of mito-ApaLI-injected embryos to blastocyst stage. Time-lapse images of EGFP reporter expression at different developmental stages. FIG. 4C is a series of representative gels of RFLP analysis and bar graphs of the quantification of mtDNA heteroplasmy in control and mito-ApaLI-injected embryos (control n=10; mito-ApaLI n=8). FIG. 4D is a bar graph and FIG. 4C is a whisker plot showing the quantification of mtDNA copy number by qPCR in control and mito-ApaLI-injected embryos (control n=18; mito-ApaLI n=12). Error bars represent±SEM. ***p<0.001. ****p<0.0001.

FIG. 5A-5B are a series of images and graphs showing the analysis of heteroplasmy shift in NZB/BALB Embryos. (FIG. 5A) RFLP analysis and quantification of mtDNA heteroplasmy in control and mito-ApaLI-injected embryos at polymorphic HindIII site (Control n=7; mito-ApaLI n=7). FIG. 5B is a bar graph and FIG. 5C is a whisker plot showing the quantification of mtDNA copy number by qPCR in control and mito-ApaLI-injected embryos at polymorphic HindIII site (Control n=5; mito-ApaLI n=6). Error bars represent±SEM. **p<0.01. ****p<0.0001.

FIG. 6A-6E is a series of illustrations, images, and graphs showing the generation of live animals after induction of heteroplasmy shift in NZB/BALB Embryos Using mito-ApaLI. FIG. 6A is an illustration showing the outline for the generation of live animals after injection of mito-ApaLI mRNA in one-cell embryos. FIG. 6B is a representative photograph of F1 mito-ApaLI mice. FIG. 6C is an image of a gel of RFLP analysis and a bar graph showing quantification of mtDNA heteroplasmy in tail tip biopsies of embryo donors and generated F1 mito-ApaLI pups. (Donor n=10; mito-ApaLI n=9). FIG. 6E is an image of a gel of RFLP analysis and quantification of mtDNA heteroplasmy in tail, brain, muscle, heart, and liver of F1 mito-ApaLI mice. FIG. 6D and FIG. 6F are each a respective bar graph showing the quantification of mtDNA copy number and levels by qPCR in F1 mito-ApaLI pups (Donor n=10; F1 mito-ApaLI n=9). Error bars represent±SEM. ****p<0.0001.

FIG. 7A-7C are a series of images and illustrations showing the analysis of F1 Mito-ApaLI Mice, related to FIG. 6. FIG. 7A is an image of a gel from a RFLP analysis of mtDNA heteroplasmy in tail tip biopsies of F1 mito-ApaLI pups at polymorphic HindIII site. (mito-ApaLI n=9). FIG. 7B is an image of a gel from a RFLP analysis of mtDNA heteroplasmy in tail, brain, muscle, heart, and liver of F1 mito-ApaLI mice at polymorphic HindIII site. FIG. 7C is an illustration of an array comparative genomic hybridization (array CGH) in mito-ApaLI mice for genome-wide detection of copy-number variants (CNVs), duplications/deletions, unbalanced translocations and aneuploidies. Gains are drawn on the right and losses on the left side of the diagram.

FIG. 8A-8F are a series of graphs and images showing the characterization of F1 mito-ApaLI mice. FIG. 8A is a scatter plot showing body weight of mito-ApaLI males (control n=5 and mito-ApaLI n=3) and mito-ApaLI females (control n=5 and mito-ApaLI n=6) at different time points. ns, non-significant. FIG. 8B and FIG. 8C are a series of bar graphs showing biochemical analysis of glucose and lactate in blood of control (n=10) and mito-ApaLI (n=9) mice. ns, non-significant. FIG. 8D-FIG. 8F area a series of bar graphs showing open field test measuring baseline levels of locomotor activity in freely moving mice quantifying respectively distance traveled, ambulatory counts, and vertical counts. FIG. 8G and FIG. 8 are a series of bar graphs showing rotarod test evaluating locomotor coordination based on the latency at which a fall occurs on a gradually accelerating spinning rod. FIG. 8I and FIG. 8J are a series of bar graphs showing grip strength test measuring average and maximum grip force in the forelimbs. FIG. 8K is an image of a gel from a RFLP analysis and bar graph showing the quantification of mtDNA heteroplasmy in tail tip biopsies of F2 mito-ApaLI pups. (F2 mito-ApaLI n=12). Error bars represent±SEM. See also FIG. 9 and TABLE 1. FIG. 8L is a whisker plot.

FIG. 9 is an image of a gel showing the analysis of F2 Mito-ApaLI mice, related to FIG. 8. RFLP analysis of mtDNA heteroplasmy in tail tip biopsies of F2 mito-ApaLI pups at the polymorphic HindIII site. (F2 mito-ApaLI n=12).

FIG. 10A-10H are a series of illustrations, graphs, and images showing induction of heteroplasmy shift in NZB/BALB tail tip fibroblasts and oocytes using NZB mito-TALEN (related to FIG. 11). FIG. 10A is a diagram illustrating targeting sequences of NZB TALEN. FIG. 10B is a schematic representation of luciferase-based NZB targeting specificity assay. FIG. 10C is a bar graph showing NZB targeting specificity of NZB TALEN collection. Blue square indicates NZB TALEN with highest specificity. FIG. 10D is an immunofluorescence image showing mitochondrial co-localization of NZB TALEN monomers with Mitotracker. Scale bars, 10 μm. FIG. 10E is a n image of gel from a RFLP analysis of mtDNA heteroplasmy in NZB/BALB cells transfected with mito-GFP and NZB TALEN after 72 hr. FIG. 10F is a bar graph showing quantification of mtDNA levels by qPCR in NZB/BALB cells transfected with mito-GFP and NZB TALEN after 72 hr. FIG. 10G is an image of a gel from an RFLP analysis and a bar graph showing the quantification of heteroplasmy shift in NZB/BALB in control and NZB TALEN-injected MII oocytes after 48 hr at the polymorphic HindIII site (control n=4; mito-ApaLI n=4). Error bars represent±SEM. *p<0.05. FIG. 10H is a bar graph.

FIG. 11A-11E are a series of images, illustrations, and bar graphs showing heteroplasmy shift in NZB/BALB MII oocytes using NZB Mito-TALEN. FIG. 11A is an illustration showing the injection of NZB mito-TALEN mRNA in oocytes for induction of heteroplasmy shift. FIG. 11B are a series of immunofluorescence images showing expression of fluorescent reporters of NZB TALEN monomer in MII oocytes. FIG. 11C is an image of gels from an RFLP analysis and a bar graph showing the quantification of mtDNA heteroplasmy in control and NZB TALEN-injected oocytes after 48 hr (control n=9; NZB TALEN n=7).

FIG. 11D is a bar graph and FIG. 11E is a whisker plot showing the quantification of mtDNA copy number by qPCR in control and NZB TALEN-injected oocytes after 48 hr (control n=16; NZB TALEN n=8). Error bars represent±SEM. **p<0.01. ***p<0.001. See also, e.g., FIG. 10.

FIG. 12A-12F is a series of illustrations, images, and graphs showing specific elimination of mtDNA with human LHOND m.14459G>A and NARP m.9176T>C mutations in mammalian oocytes using mito-TALENs. FIG. 12A is an illustration of the fusion of human cells harboring LHOND m.14459G>A and NARP m.9176T>C mutations with mouse MII oocytes followed by the injection of mito-TALENs for induction of heteroplasmy shift.

FIG. 12B are a series of representative images of MII oocytes before and after cell fusion. FIG. 12C is an image of a gel from a RFLP analysis and quantification of LHOND heteroplasmy in individual MII oocytes with and without LHOND TALEN injection after 48 hr (fusion n=3; Fusion+TALEN n=3). FIG. 12D is a whisker plot showing the quantification of human mtDNA copy number by qPCR in individual MII oocytes with and without LHOND TALEN injection after 48 hr (fusion n=4; fusion+TALEN n=4). FIG. 12E is an image of a gel from a RFLP analysis and quantification of NARP heteroplasmy in individual MII oocytes with and without NARP TALEN injection after 48 hr (fusion n=7; fusion+TALEN n=3). FIG. 12F is a whisker plot showing the quantification of human mtDNA copy number by qPCR in individual MII oocytes with and without NARP TALEN injection after 48 hr (fusion n=17; fusion+TALEN n=9). Error bars represent±SEM. *p<0.05. ***p<0.001. See also, e.g., FIG. 13.

FIG. 13A-13I is a series of illustrations, images, and graphs showing induction of heteroplasmy shift using patient specific TALENS, related to FIG. 12. FIG. 13A is an image of a gel from PCR analysis of human mtDNA region containing the LHON m.14459G>A mutation in fused MII oocytes. FIG. 13B is a series of immunofluorescence images showing the expression of fluorescent reporters of LHOND TALEN monomer in fused MII oocytes. FIG. 13C is a diagram illustrating targeting sequences of NARP TALEN. FIG. 13D is a schematic representation of luciferase-based NARP targeting specificity assay. FIG. 13E is a bar graph showing NARP targeting specificity of NARP TALEN collection. Dashed square indicates NARP TALEN with highest specificity. FIG. 13F is a series of immunofluorescence images showing mitochondrial co-localization of NARP mito-TALEN monomers with Mitotracker. Scale bars, 10 mm. FIG. 13G is a gel from a RFLP analysis of mtDNA heteroplasmy in NARP patient cells transfected with mito-GFP and NARP TALEN after 72 hr. FIG. 13H is a bar graph showing quantification of mtDNA levels by qPCR in NARP patient cells transfected with mito-GFP and NARP TALEN after 72 hr. FIG. 13I is a series of immunofluorescence images showing expression of fluorescent reporters of NARP TALEN monomer in fused MII oocytes. Error bars represent±SEM.

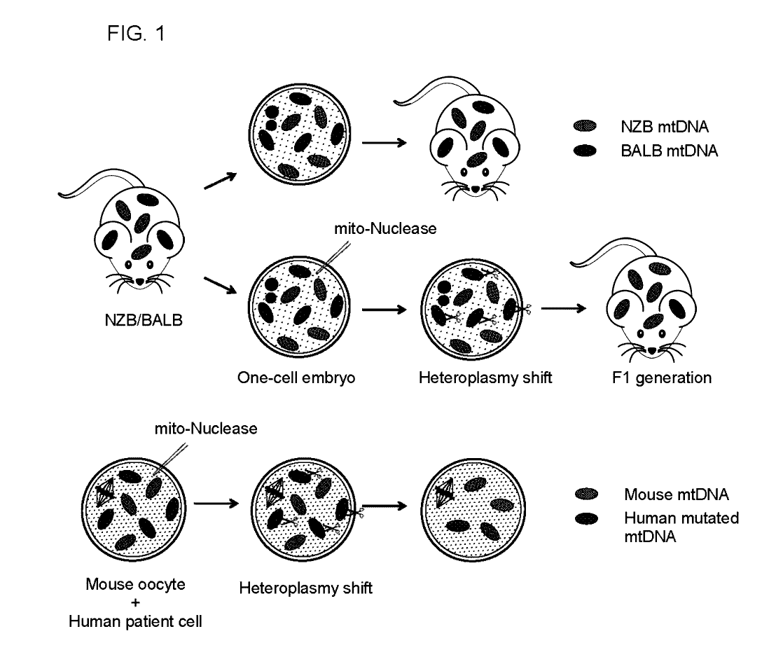

### DETAILED DESCRIPTION OF THE INVENTION

The present disclosure is based, at least in part, on the discovery that mitochondria-targeted nucleases injected into oocytes or embryos can reduce the number of mitochondrial DNA (mtDNA) haplotypes in a germline, which can prevent the transgenerational transmission of mutated mtDNA or allow for systemic clearance of mutated mtDNA in subsequent generations. Described herein are methods of preventing transgenerational transmission of a mitochondria-associated disease, disorder, or condition. Nucleases or mRNA can be introduced (e.g., injected) into oocytes or embryos, which can prevent transgenerational transmission of a mitochondria-associated disease, disorder, or condition.

Further described herein are mitochondria-targeted nucleases that can selectively reduce mtDNA haplotypes in germline. The germline heteroplasmy shift can prevent transgenerational transmission of mtDNA haplotypes to offspring. Methods described herein can use mitochondria-targeted nucleases or RNA to reduce mutated mtDNA in human oocytes by preventing transgenerational transmission of mtDNA haplotypes to offspring.

Provided herein are alternative or complementary clinical paths that can alleviate or eliminate ethical concerns to prevent the transmission of mitochondrial diseases caused by mtDNA mutations. Previous studies failed to provide mechanisms for preventing transgenerational transmission of mtDNA haplotypes to offspring transmission of mutated mtDNA nor did previous studies allow for a substantially complete or complete systemic clearance of mutated mtDNA in subsequent generations.

Also provided herein are methods of preventing transgenerational transmission of mutated mitochondrial genome associated with neurodegenerative disease, diabetes, cardiomyopathy, cancer, or aging.

Mitochondria and Mitochondrial DNA (MTDNA)

Mitochondria are understood to be double-membrane cellular organelles of bacterial origin that play a role in multiple processes including energy production, calcium homeostasis, cellular signaling, and apoptosis (Dyall et al., 2004). Mitochondria contain their own mtDNA encoding 13 polypeptides of the mitochondrial respiratory chain as well as tRNAs or rRNAs necessary for their synthesis (Anderson et al., 1981). mtDNA can be present in multiple copies per cell, ranging from approximately 1,000 copies in somatic cells to several 100,000 copies in oocytes, with an average 1-10 copies per organelle (Shoubridge and Wai, 2007). In contrast to nuclear DNA, mtDNA can be exclusively transmitted through maternal inheritance. Conventional methods for the study mitochondrial structure, function, and biogenesis are well-known (see e.g., Pone et al. (2011) Mitochondria Meth in Cell Biol. 80, ISBN-13: 978-0470040737). Except as otherwise noted herein, therefore, a process of the present disclosure can be carried out in accordance with such processes.

Due to the thousands of copies of mtDNA contained within a cell, the levels of mutated mtDNA can vary, as described herein. The term homoplasmy can refer to the presence of a single mtDNA haplotype in the cell, whereas heteroplasmy can refer to the coexistence of more than one mtDNA haplotype. A haplotype can be a set of DNA variations, or polymorphisms, that tend to be inherited together. A haplotype can refer to a combination of alleles or to a set of single nucleotide polymorphisms (SNPs) found on the same chromosome.

As described herein, a disease state may ensue if a percentage of mutated mtDNA molecules exceeds a threshold that compromises mitochondrial function (Taylor and Turnbull, 2005; Wallace and Chalkia, 2013). For example, threshold levels for biochemical and clinical defects can be in the range of about 60% to about 95% mutated mtDNA depending on the severity of the mutation (Russell and Turnbull, 2014). As another example, the threshold levels for biochemical and clinical defects can be in the range of about 6% to about 100% mutated mtDNA. As another example, threshold levels for biochemical or clinical defects can be about 6% mutated mtDNA; about 7% mutated mtDNA; about 8% mutated mtDNA; about 9% mutated mtDNA; about 10% mutated mtDNA; about 11% mutated mtDNA; about 12% mutated mtDNA; about 13% mutated mtDNA; about 14% mutated mtDNA; about 15% mutated mtDNA; about 16% mutated mtDNA; about 17% mutated mtDNA; about 18% mutated mtDNA; about 19% mutated mtDNA; about 20% mutated mtDNA; about 21% mutated mtDNA; about 22% mutated mtDNA; about 23% mutated mtDNA; about 24% mutated mtDNA; about 25% mutated mtDNA; about 26% mutated mtDNA; about 27% mutated mtDNA; about 28% mutated mtDNA; about 29% mutated mtDNA; about 30% mutated mtDNA; about 31% mutated mtDNA; about 32% mutated mtDNA; about 33% mutated mtDNA; about 34% mutated mtDNA; about 35% mutated mtDNA; about 36% mutated mtDNA; about 37% mutated mtDNA; about 38% mutated mtDNA; about 39% mutated mtDNA; about 40% mutated mtDNA; about 41% mutated mtDNA; about 42% mutated mtDNA; about 43% mutated mtDNA; about 44% mutated mtDNA; about 45% mutated mtDNA; about 46% mutated mtDNA; about 47% mutated mtDNA; about 48% mutated mtDNA; about 49% mutated mtDNA; about 50% mutated mtDNA; about 51% mutated mtDNA; about 52% mutated mtDNA; about 53% mutated mtDNA; about 54% mutated mtDNA; about 55% mutated mtDNA; about 56% mutated mtDNA; about 57% mutated mtDNA; about 58% mutated mtDNA; about 59% mutated mtDNA; about 60% mutated mtDNA; about 61% mutated mtDNA; about 62% mutated mtDNA; about 63% mutated mtDNA; about 64% mutated mtDNA; about 65% mutated mtDNA; about 66% mutated mtDNA; about 67% mutated mtDNA; about 68% mutated mtDNA; about 69% mutated mtDNA; about 80% mutated mtDNA; about 81% mutated mtDNA; about 82% mutated mtDNA; about 83% mutated mtDNA; about 84% mutated mtDNA; about 85% mutated mtDNA; about 86% mutated mtDNA; about 87% mutated mtDNA; about 88% mutated mtDNA; about 89% mutated mtDNA; about 90% mutated mtDNA; about 91% mutated mtDNA; about 92% mutated mtDNA; about 93% mutated mtDNA; about 94% mutated mtDNA; about 95% mutated mtDNA; about 96% mutated mtDNA; about 97% mutated mtDNA; about 98% mutated mtDNA; about 99% mutated mtDNA; or about 100% mutated mtDNA. It is understood that recitation of the above discrete value includes a range between each recited value.

Changes in the relative levels of heteroplasmic mtDNA can be referred to as mtDNA heteroplasmy shifts. Despite the fact that mitochondria can possess all the necessary machinery for homologous recombination and non-homologous end joining, it is not currently believed they represent the major pathway for mtDNA repair in mammalian mitochondria (Alexeyev et al., 2013).

### Mitochondria-Associated Disease, Disorder, Dysfunction, or Condition

As described herein, a mitochondria-associated disease can be any mitochondria-associated disease, disorder, dysfunction, or condition. For example, a mitochondria-associated disease can be any disease, disorder, dysfunction, or condition caused by a mtDNA mutation.

A mitochondrial-associated disease can include a maternally inherited genetic disorder caused in whole or in part by mutations in mtDNA. In many subjects with a mitochondria-associated disease, mutated mtDNA can coexist with wild-type mtDNA, a situation known as mtDNA heteroplasmy.

Amitochondrial disease can be the result of inherited mutations or spontaneous mutations in mtDNA, which can lead to altered functions of the proteins or RNA molecules that normally reside in mitochondria.

As understood in the art, mitochondria play a role in energy production, calcium homeostasis, cellular signaling, or apoptosis. Because mitochondria can play a role in energy production, mitochondrial diseases can correlate with degeneration of tissues or organs with high-energy demands. For example, mitochondria-associated diseases can lead to myopathies, cardiomyopathies, or encephalopathies, among other phenotypes (Taylor and Turnbull, 2005). Mitochondrial-associated diseases or disorders can cause damage to cells of the brain, heart, liver, skeletal muscles, kidney, the endocrine system, or respiratory system. Mitochondria-associated disease include, but are not limited to, Neuropathy ataxia retinitis pigmentosa (NARP) syndrome; mitochondrial encephalo-myopathy with lactic acidosis or stroke like episodes (MELAS); maternally inherited Leigh's syndrome (MILS) syndrome; NARP-MILS syndrome; hemolytic anemia; a neurodegenerative disease; diabetes; mitochondrial dysfunction-associated aging; gastro-intestinal disorders; cardiac disease; liver disease; diabetes; respiratory disease; seizures; visual/hearing loss; lactic acidosis; developmental delays; mitochondrial myopathy; diabetes mellitus; diabetes mellitus and deafness (DAD); Leber's hereditary optic neuropathy (LHON); Leber's hereditary optic neuropathy and dystonia (LHOND); Wolff-Parkinson-White syndrome; multiple sclerosis-type disease; Leigh syndrome; subacute sclerosing encephalopathy; seizures; altered states of consciousness; dementia; ventilatory failure; neuropathy, ataxia, retinitis pigmentosa, and ptosis (NARP); myoneurogenic gastrointestinal encephalopathy (MNGIE); gastrointestinal pseudo-obstruction; neuropathy; Myoclonic Epilepsy with Ragged Red Fibers (MERRF); progressive myoclonic epilepsy; hearing loss; Mitochondrial myopathy; encephalomyopathy; lactic acidosis; mtDNA depletion; mitochondrial neurogastrointestinal encephalomyopathy (MNGIE); or Friedreich's ataxia. Other mitochondria-associated diseases may be known in the art or later discovered, all of which can benefit from the present disclosure.

Because mtDNA mutations in the germline have been recently linked to aging (Ross et al., 2013), methods and compositions as described herein can also be applied to prevent the transmission of mtDNA variants with potential roles in aggravating aspects of human aging or age-associated diseases.

An exemplary mitochondria-associated diseases is Leigh syndrome. Leigh syndrome may be caused by the NARP mutation, the MERRF mutation, complex I deficiency, cytochrome oxidase (COX) deficiency, pyruvate dehydrogenase (PDH) deficiency, or other unmapped DNA changes. Methods and compositions disclosed herein can be used in the treatment of Leigh syndrome.

Female Gametocyte, Germ Cell, or Embryo

As described herein, mRNA encoding an enzyme (e.g., a restriction enzyme) an be introduced to a female gametocyte, germ cell, or embryo to prevent the transmission of a mutated mtDNA. A gametocyte can be a eukaryotic germ cell or an oocyte (also known as oöcyte, ovocyte, or ocyte) involved in reproduction. An oocyte can be an immature ovum, or egg cell. An oocyte can be produced in the ovary during female gametogenesis. The female germ cells can produce a primordial germ cell (PGC), which then undergoes mitosis, forming oogonia. During oogenesis, the oogonia can become a primary oocyte. The formation of an oocyte can be called oocytogenesis, which can be a part of oogenesis. An oocyte can be primary or secondary. Oogenesis can result in the formation of both primary oocytes before birth, or of secondary oocytes after it as part of ovulation. Various compositions described herein can be introduced into the above described structures or can prevent transmission of a mutated mtDNA.

Mitochondria-Targeted Enzyme or Nuclease

The methods as described herein can employ a mitochondria-targeted enzyme or mitochondria-targeted nuclease (e.g., mitochondria-targeted restriction enzyme) or mRNA that encodes a mitochondria-targeted enzyme or mRNA that encodes a mitochondria-targeted nuclease (e.g., mRNA that encodes a mitochondria-targeted restriction enzyme). A mitochondria-targeted enzyme (or mitochondria-targeted nuclease) can be any conventional mitochondria-targeted enzyme or can be any mRNA encoding such a mitochondria-targeted enzyme.

A nuclease or enzyme can be delivered is any form known in the art. For example, a nuclease or enzyme can be introduced with mRNA encoding for a nuclease or enzyme. As another example, a nuclease or enzyme can be introduced in protein form.

The terms mitochondria-targeted nuclease, mitochondria-targeted enzyme, mitochondria-targeted restriction enzyme, mitochondria-targeted restriction nuclease, mRNA encoding a mitochondria-targeted enzyme, mRNA encoding a mitochondria-targeted nuclease, mRNA encoding a mitochondria-targeted restriction enzyme, mRNA encoding a mitochondria-targeted restriction nuclease can be interchangeable.

A restriction enzyme (e.g., restriction nuclease, restriction endonuclease) can be an enzyme that cuts DNA at or near specific recognition nucleotide sequences known as a restriction site. Such restriction enzymes can be classified into three types, which differ in their structure and whether they cut their DNA substrate at their recognition site, or if the recognition and cleavage sites are separate from one another. Such restriction enzymes can cut DNA by making two incisions, once through each sugar-phosphate backbone (i.e. each strand) of the DNA double helix.

As described herein, a restriction enzyme can recognize a specific sequence of nucleotides or can produce a double-stranded cut in the DNA. Such recognition sequences can also be classified by the number or bases in its recognition site, which can typically be between 4 and 8 bases, and the amount of bases in the sequence can determine how often the site will appear by chance in any given genome (e.g., a 4 base pair sequence would theoretically occur once every 4⁴ or 256 bp, 6 bases, 4⁶ or 4,096 bp, and 8 bases would be 4⁸ or 65,536 bp). For example, many such base pair sequences can be palindromic, meaning the base sequence reads the same backwards and forwards. It is presently thought there are two types of palindromic sequences that can be possible in DNA. For example, the mirror-like palindrome is similar to those found in ordinary text, in which a sequence reads the same forward and backwards on a single strand of DNA strand, as in GTAATG. As another example, the inverted repeat palindrome is a sequence that reads the same forward and backwards, but the forward and backward sequences are found in complementary DNA strands (i.e., of double-stranded DNA), as in GTATAC (GTATAC being complementary to CATATG). Inverted repeat palindromes can be more common or can have greater biological importance than mirror-like palindromes.

Because recognition sequences in DNA can differ for each restriction enzyme (producing differences in the length, sequence, or strand orientation (5′ end or the 3′ end) of a sticky-end “overhang” of an enzyme restriction), restriction enzymes (or mRNA encoding restriction enzymes) can be designed for the recognition of specific DNA sequences. For example, different restriction enzymes that recognize the same sequence are known as neoschizomers. These often cleave in different locales of the sequence or different enzymes that recognize or cleave in the same location are known as isoschizomers.

As described herein, natural restriction enzymes or artificial (e.g., synthetic) restriction enzymes (or mRNA encoding natural restriction enzymes or synthetic restriction enzymes) can be employed. Naturally occurring restriction endonucleases can be categorized into four groups (Types I, II III, or IV) based on their composition and enzyme cofactor requirements, the nature of their target sequence, or the position of their DNA cleavage site relative to the target sequence. Enzymes can recognize specific short DNA sequences or carry out the endonucleolytic cleavage of DNA to give specific fragments with terminal 5′-phosphates. But types of enzymes can differ in their recognition sequence, subunit composition, cleavage position, or cofactor requirements, as described below.

For example, Type I enzymes can cleave at sites remote from recognition site; require both ATP and S-adenosyl-L-methionine to function; multifunctional protein with both restriction and methylase activities. As another example, Type II enzymes can cleave within or at short specific distances from recognition site; most require magnesium; or are single function (restriction) enzymes independent of methylase. As another example, Type III enzymes can cleave at sites a short distance from recognition site; require ATP (but do not hydrolyse it); are stimulated by S-adenosyl-L-methionine but such factor is not required; or exist as part of a complex with a modification methylase. As another example, Type IV enzymes can target modified DNA (e.g. methylated, hydroxymethylated or glucosyl-hydroxymethylated DNA).

As described herein, artificial or synthetic restriction enzymes can be employed. For example, artificial restriction enzymes can be generated by fusing a natural or engineered DNA binding domain to a nuclease domain (often the cleavage domain of the type IIS restriction enzyme Fokl.) Such artificial restriction enzymes can target large DNA sites (e.g., up to 36 bp) or can be engineered to bind to desired DNA sequences. As described herein, zinc finger nucleases can be used as an artificial restriction enzymes. Zinc finger nucleases can be generally used in genetic engineering applications or can also be used for more standard gene cloning applications. Other artificial restriction enzymes can be based on the DNA binding domain of TAL effectors.

As described herein, isolated restriction enzymes can be used to manipulate DNA. For example, restriction enzymes can be used to assist insertion of genes into plasmid vectors during gene cloning or protein expression experiments. As described herein, plasmids used for gene cloning can be modified to include a short polylinker sequence (e.g., the multiple cloning site, or MCS) rich in restriction enzyme recognition sequences. This can allow for flexibility when inserting gene fragments into the plasmid vector. Restriction sites contained naturally within genes can influence the choice of endonuclease for digesting the DNA because it can be advantageous to avoid restriction of desired DNA while intentionally cutting the ends of the DNA. To clone a gene fragment into a vector, both plasmid DNA and gene insert can be cut with the same restriction enzymes, and then glued together with the assistance of an enzyme known as a DNA ligase.

As described herein, restriction enzymes can also be used to distinguish gene alleles by specifically recognizing single base changes in DNA known as single nucleotide polymorphisms (SNPs), which can be possible if a SNP alters the restriction site present in the allele. In such a method, the restriction enzyme can be used to genotype a DNA sample without the need for expensive gene sequencing. The sample can be first digested with the restriction enzyme to generate DNA fragments, and then the different sized fragments can be separated by gel electrophoresis. In general, alleles with correct restriction sites will generate two visible bands of DNA on the gel, and those with altered restriction sites will not be cut and will generate only a single band. The number of bands can reveals the sample subject's genotype, an example of restriction mapping.

Similarly, such methods using restriction enzymes can be used to digest genomic DNA for gene analysis by Southern blot. This technique can allow researchers to identify how many copies (or paralogues) of a gene are present in the genome of one individual, or how many gene mutations (polymorphisms) have occurred within a population. The latter example is called restriction fragment length polymorphism (RFLP).

As described herein, artificial restriction enzymes can be created by linking the Fokl DNA cleavage domain with an array of DNA binding proteins or zinc finger arrays, denoted zinc finger nucleases (ZFN). Such restriction enzymes can be a powerful tool for host genome editing due to their enhanced sequence specificity. ZFN can work in pairs, wherein their dimerization being mediated in-situ through the Fokl domain. Each zinc finger array (ZFA) can be capable of recognizing 9-12 base-pairs, making for 18-24 for the pair. A 5-7 bp spacer between the cleavage sites can further enhance the specificity of ZFN, making them a safe and more precise tool that can be applied in humans.

As described herein, restriction enzymes can be found in bacteria or archaea and provide a defense mechanism against invading viruses. Inside a prokaryote, the restriction enzymes selectively cut up foreign DNA in a process called restriction; while host DNA is protected by a modification enzyme (a methyltransferase) that modifies the prokaryotic DNA and blocks cleavage. Together, these two processes form the restriction modification system.

As described herein, mitochondria-targeted restriction enzyme or mRNA encoding a mitochondria-targeted restriction enzyme can be designed against virtually any mutation. Design can be according to techniques understood in the art (see e.g., Bacman et al., 2013; Gammage et al., 2014; Minczuk et al., 2006; 2008).

In some embodiments, induction of heteroplasmic shift in oocytes or embryos using nucleases (restriction endonucleases (REs) or transcription activator-like effector nucleases (TALENs)) to selectively eliminate mutated copies of mtDNA can prevent transmission of mutated mitochondrial genomes (mtDNA).

For example, a mitochondria-targeted restriction enzyme or mRNA encoding a mitochondria-targeted restriction enzyme can include, but are not limited to: a naturally occurring restriction endonuclease Xmal, a mitochondria-targeted transcription activator-like effector nucleases (TALENs) or mito-TALENs, a zinc finger nucleases (ZFNs), mitochondria-targeted ApaLI (mito-ApaLI), HindIII, BcII, or BsrI. As another example, restriction enzymes can include, but are not limited to: EcoRI (Escherichia coli); EcoRII (Escherichia coli); BamHI (Bacillus amyloliquefaciens); HindIII (Haemophilus influenzae); TaqI (Thermus aquaticus); NotI (Nocardia otitidis); HinFI: “Hin”FI (Haemophilus influenzae); Sau3Al (Staphylococcus aureus); PvuII (Proteus vulgaris); SmaI (Serratia marcescens) HaeIII (Haemophilus aegyptius); HgaI (Haemophilus gallinarum); Alul (Arthrobacter luteus); EcoRV (Escherichia coli); EcoP15I (Escherichia coli); Kpnl (Klebsiella pneumoniae); Pstl (Providencia stuartii); SacLl (Streptomyces achromogenes); Sall (Streptomyces albus); Scal (Streptomyces caespitosus); Spel (Sphaerotilus natans); Sphl (Streptomyces phaeochromogenes); Stul (Streptomyces tubercidicus); or Xbal (Xanthomonas badrii).

In another embodiment, mRNA encoding a mitochondria-targeted restriction enzyme or mRNA encoding a mitochondria-targeted restriction enzyme can be introduced to a female gametocyte (e.g., oocyte) or embryo. For example, mRNA can encode a mitochondria-targeted restriction enzyme or nuclease.

Molecular Engineering

The following definitions and methods are provided to better define the present disclosure and to guide those of ordinary skill in the art in the practice of the present disclosure. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide or polypeptide variants having, for example, at least 95-99% identity to the reference sequence described herein and screen such for desired phenotypes according to methods routine in the art.

Nucleotide or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (sRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating or preventing mitochondria-associated diseases, disorders, or conditions in a subject. In some embodiments, a therapeutically effective amount of a mitochondria-targeted restriction enzyme, such as a nuclease (e.g., restriction endonucleases (RE), a transcription activator-like effector nuclease (TALENs)), or an mRNA encoding a restriction enzyme, can be administered to a subject so as to selectively eliminate mutated copies of mtDNA.

Methods described herein are generally performed on a subject in need thereof. A subject in need of the therapeutic methods described herein can be a subject having, diagnosed with, suspected of having, or at risk for developing a mitochondria-associated disease, disorder, or condition. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of an agent described herein (e.g., a mitochondria-targeted restriction enzyme) can be that amount causing a desired therapeutic effect in a subject while minimizing undesired side effects. In various embodiments, an effective amount of an agent described herein (e.g., a mitochondria-targeted restriction enzyme) can reduce or substantially reduce the number of mutated mtDNA copies, or prevent the transmission of a mitochondria-associated disease, disorder, or condition.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

When used in the treatments described herein, a therapeutically effective amount of an agent described herein (e.g., a mitochondria-targeted restriction enzyme) can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to, e.g., reduce the number of mutated mtDNA copies or prevent transmission of the mitochondria-associated disease.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular gametocyte will depend upon a variety of factors including the mitochondrial disease or mutated mitochondrial DNA being targeted and the severity of the disorder; activity of the specific compound employed; the specific composition employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503).

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of an agent described herein (e.g., a mitochondria-targeted restriction enzyme or a mRNA encoding a mitochondria-targeted restriction enzyme) can occur as a single event, as several events, or over a time course of treatment.

Treatment in accord with the methods described herein can be performed prior to, concurrent with, or after conventional treatment modalities for screening for a mitochondria-associated disease, disorder, or condition.

An agent described herein (e.g., a mitochondria-targeted restriction enzyme) can be administered simultaneously or sequentially with another agent. For example, a mitochondria-targeted restriction enzyme or a mRNA encoding a mitochondria-targeted restriction enzyme can be administered simultaneously with another agent. Simultaneous administration can occur through administration of separate compositions, each containing one or more of a mitochondria-targeted restriction enzyme, a mRNA encoding a mitochondria-targeted restriction enzyme, or another agent. An agent described herein (e.g., a mitochondria-targeted restriction enzyme) can be administered sequentially with another agent. For example, a mitochondria-targeted restriction enzyme can be administered before or after administration of another agent.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to mitochondria-targeted restriction enzymes, mRNA encoding mitochondria-targeted restriction enzymes, qPCR kits, plasmids, RFLP analysis kits, or fluorescent imaging stains or dyes. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### Overview

The following examples describe a strategy for preventing germline transmission of mitochondrial diseases by inducing mitochondrial DNA (mtDNA) heteroplasmy shift and specific reduction of mutated mitochondrial genomes through the selective elimination of mutated mtDNA.

Mutated mitochondrial genomes responsible for human mitochondrial diseases were successfully reduced in mouse oocytes using mitochondria-targeted nucleases. For example, successful reduction in human mutated mtDNA levels responsible for Leber's hereditary optic neuropathy (LHOND), and neurogenic muscle weakness, ataxia, and retinitis pigmentosa (NARP), in mammalian oocytes was achieved using mitochondria-targeted TALEN (mito-TALENs). Altogether, the approaches described represent a therapeutic avenue for preventing the transgenerational transmission of human mitochondrial diseases caused by mutations in mtDNA.

Further, by taking advantage of NZB/BALB heteroplasmic mice (which contain two mtDNA haplotypes, BALB and NZB), it was shown that their germline transmission was selectively prevented using either mitochondria-targeted restriction endonucleases or TALENs. It was shown that BALB or NZB mitochondrial genomes in the germline were specifically reduced in the heteroplasmic NZB/BALB mouse model using mitochondria-targeted restriction endonucleases and TALENs and also prevented their transmission to the next generation.

The approaches presented in the following examples may be applied and developed to prevent the transgenerational transmission of human mitochondrial diseases.

### Example 2

### Specific Reduction of Mitochondrial Genomes in Oocytes and Embryos Using Restriction Endonucleases

The following example describes the specific reduction of mitochondrial genomes in oocytes and embryos using restriction endonucleases as a therapeutic approach for preventing the germline transmission of mitochondrial diseases caused by mtDNA mutations by selectively eliminating BALB mtDNA in NZB/BALB oocytes.

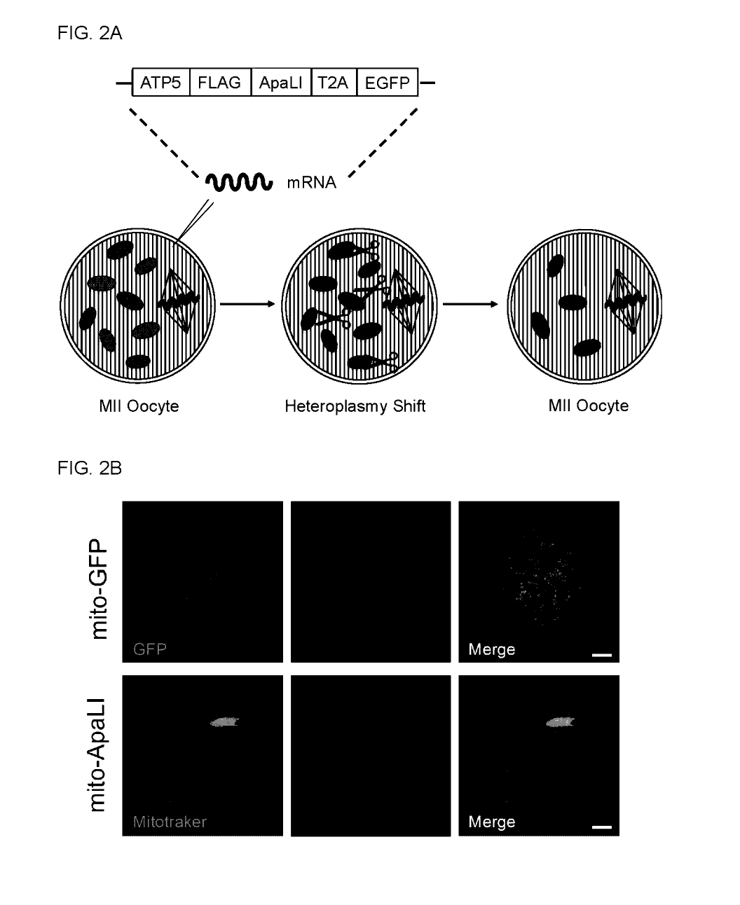

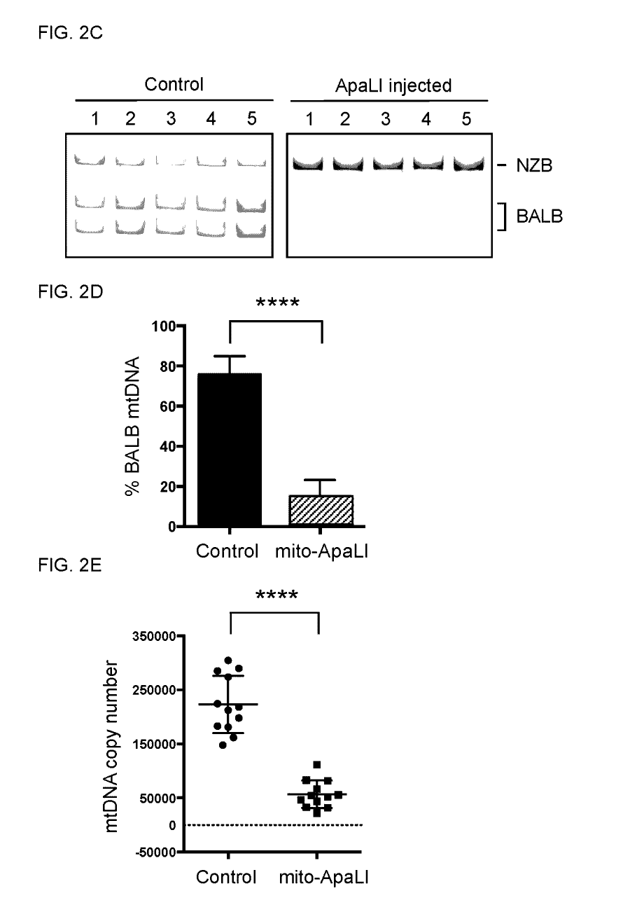

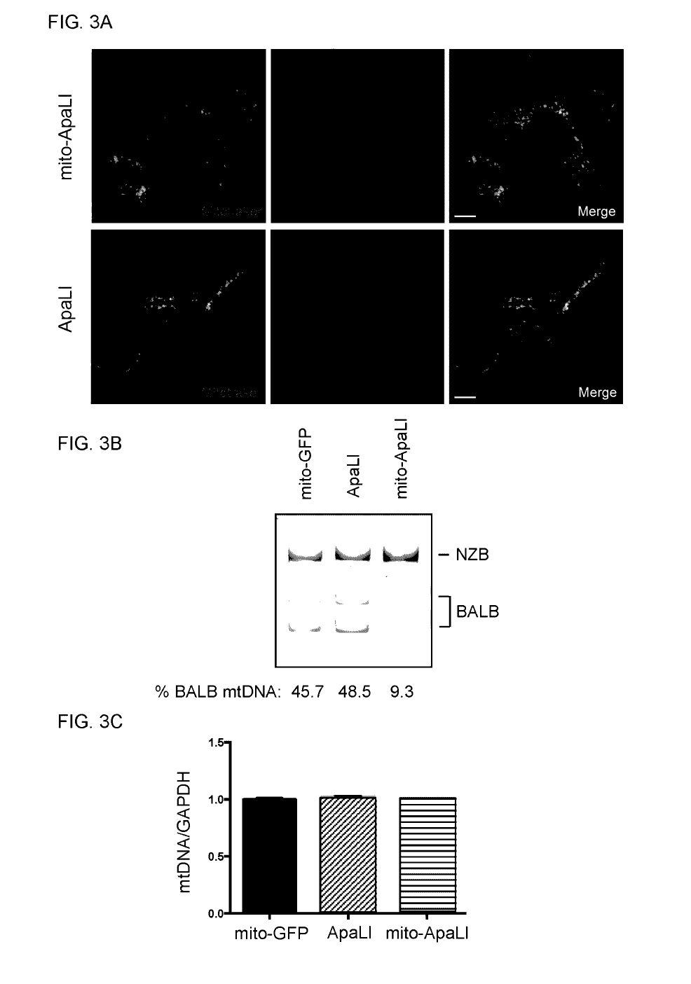

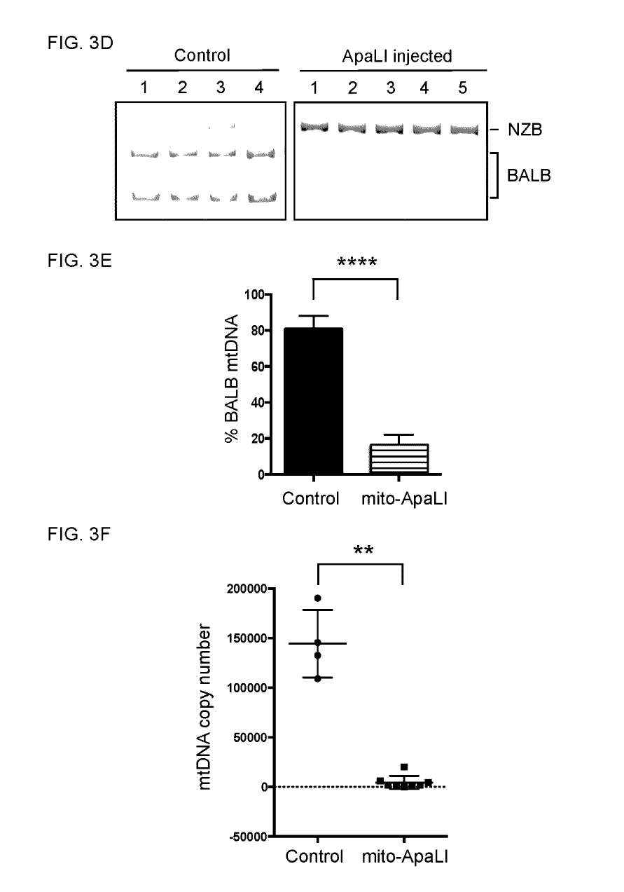

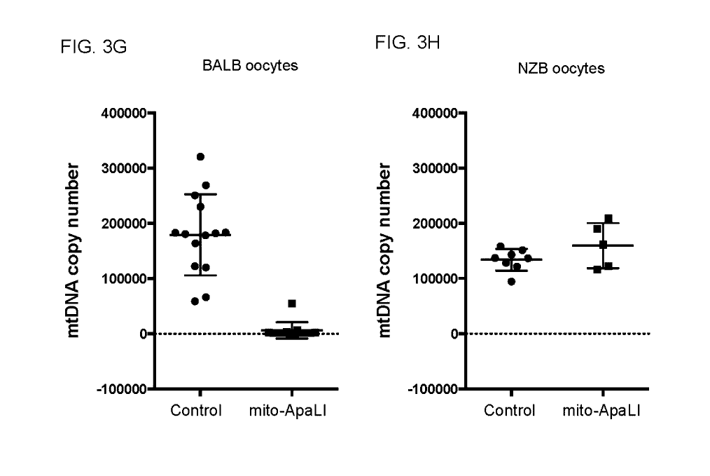

The specific elimination of BALB mtDNA in NZB/BALB oocytes and one-cell embryos were tested. For this purpose, a mammalian codon optimized ApaLI targeted to mitochondria by the ATP5b mitochondria targeting sequence and the ATP5b 5′ and 3′ UTRs to promote co-translation import from mitochondrial associated ribosomes was generated. An enhanced GFP (EGFP) reporter was also included in the construct to monitor expression (see e.g., FIG. 2A). First, the mitochondrial localization of the ApaLI protein generated from the construct by immunostaining in NZB/BALB tail tip fibroblasts (TTFs) was tested and robust co-localization of mitochondria-targeted ApaLI (mito-ApaLI) was observed with the mitochondrial dye Mitotracker (see e.g., FIG. 3A). In contrast, mitochondrial localization of non-mitochondria-targeted ApaLI was not observed (see e.g., FIG. 3A). Analysis of mtDNA by “lastcycle hot” PCR and restriction fragment length polymorphism (RFLP) demonstrated induction of heteroplasmy shift by specific reduction of BALB mtDNA in cells transfected with mito-ApaLI compared to control cells transfected with mito-GFP after 72 hr (see e.g., FIG. 3B). In addition, normal mtDNA copy number was found in mito-ApaLI transfected cells, which resulted from the replication of the remaining NZB mtDNA that compensated for the reduction of BALB mtDNA (see e.g., FIG. 3C).

Next, it was tested whether a similar approach could be used in oocytes to specifically eliminate BALB mtDNA (see e.g., FIG. 2A). First, the mitochondrial localization of mito-ApaLI was confirmed in NZB/BALB metaphase II (MII) oocytes injected with mRNA encoding mito-ApaLI by immunostaining (see e.g., FIG. 2B). As expected, mito-ApaLI co-localized with Mitotracker in MII oocytes (see e.g., FIG. 2B). RFLP analysis 48 hr after mito-ApaLI mRNA injection demonstrated the specific reduction of BALB mtDNA and a consequential increase in the relative NZB mtDNA levels (see e.g., FIG. 2C). In agreement with the lack of mtDNA replication in mature oocytes and pre-implantation embryos (Wai et al., 2010), analysis of mtDNA copy number by qPCR revealed a decrease in mtDNA copy number following mito-ApaLI injection proportional to the initial levels of BALB mtDNA (see e.g., FIG. 2D). To verify the reduction of BALB mtDNA, RFLP and qPCR analyses was performed by amplification of an independent region of the mtDNA containing a unique HindIII site, exclusively present in BALB mtDNA. These analyses confirmed the specific reduction of BALB mtDNA upon injection of mito-ApaLI in NZB/BALB MII oocytes (see e.g., FIG. 3D and FIG. 3E). Injection of mito-ApaLI in BALB or NZB single haplotype oocytes resulted in complete depletion of mtDNA in BALB oocytes and did not affect mtDNA levels in NZB oocytes reinforcing the specificity of mito-ApaLI (see e.g., FIG. 3F). Collectively, these results suggest this approach can specifically reduce mtDNA in the germline.

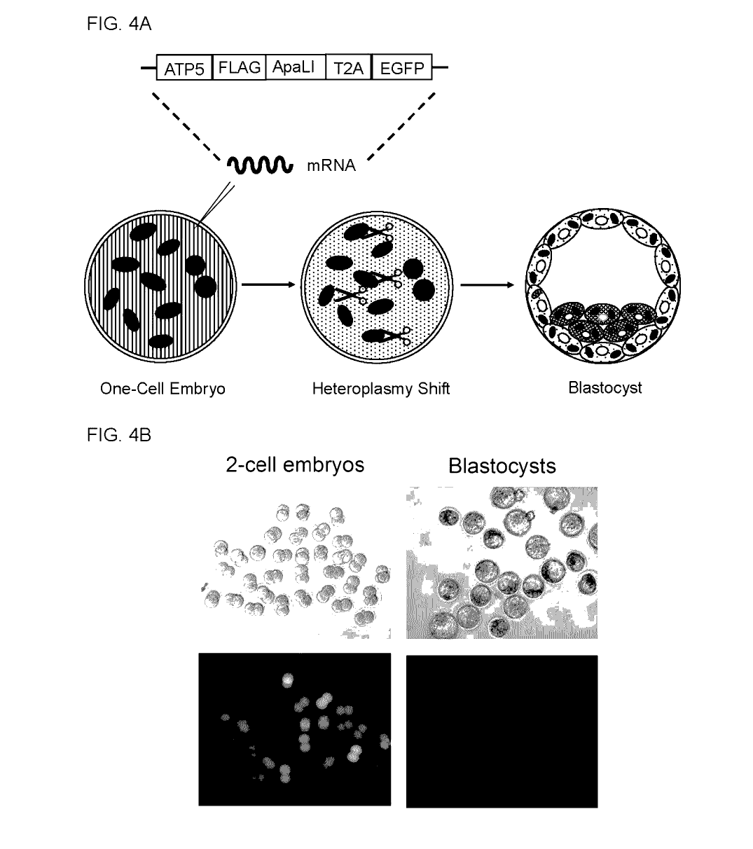

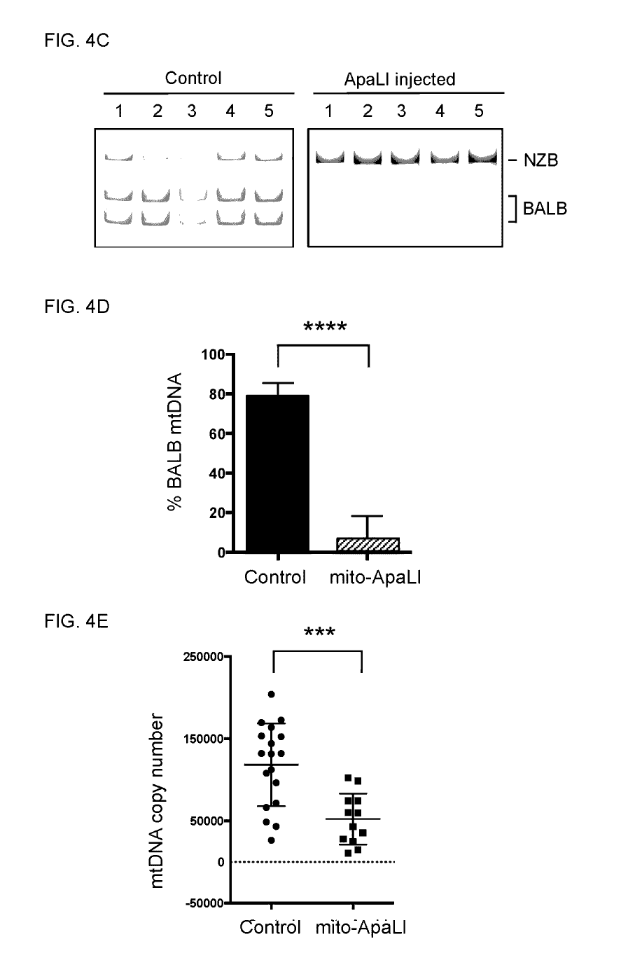

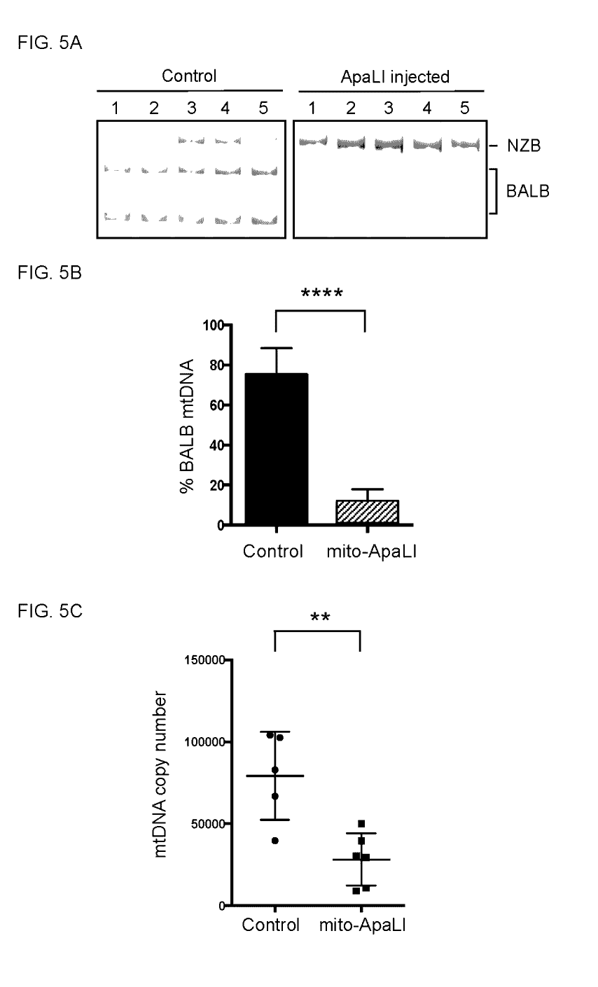

In addition to oocytes, mtDNA heteroplasmy shift was tested to determine if it could be applied to one-cell embryos without affecting their normal development until the blastocyst stage (see e.g., FIG. 4A). For this purpose, NZB/BALB one-cell embryos were injected with mito-ApaLI mRNA. Time-lapse fluorescent microscopy images revealed the expression of mito-ApaLI indicated by EGFP expression, and more importantly, normal development of mito-ApaLI-injected embryos through the different developmental stages analyzed (see e.g., FIG. 4B). Similar to the results observed in oocytes, RFLP analysis of mito-ApaLI blastocysts demonstrated specific reduction of BALB mtDNA and an increase in the relative levels of NZB mtDNA (see e.g., FIG. 4C). Moreover, due to the lack of mtDNA replication until the blastocyst stage (Wai et al., 2010), analysis of mtDNA copy number by qPCR showed a decrease in mtDNA levels proportional to the BALB mtDNA levels (see e.g., FIG. 4D). RFLP and qPCR analyses at the HindIII region confirmed the specific reduction of BALB mtDNA upon injection of mito-ApaLI in NZB/BALB embryos (see e.g., FIG. 5A and FIG. 5B).

### Example 3

### Preventing the Transmission of Mitochondrial Genomes Using Mitochondria-Targeted Restriction Endonucleases

The following example describes the prevention of transmission of mitochondrial genomes using mitochondria-targeted restriction endonucleases.

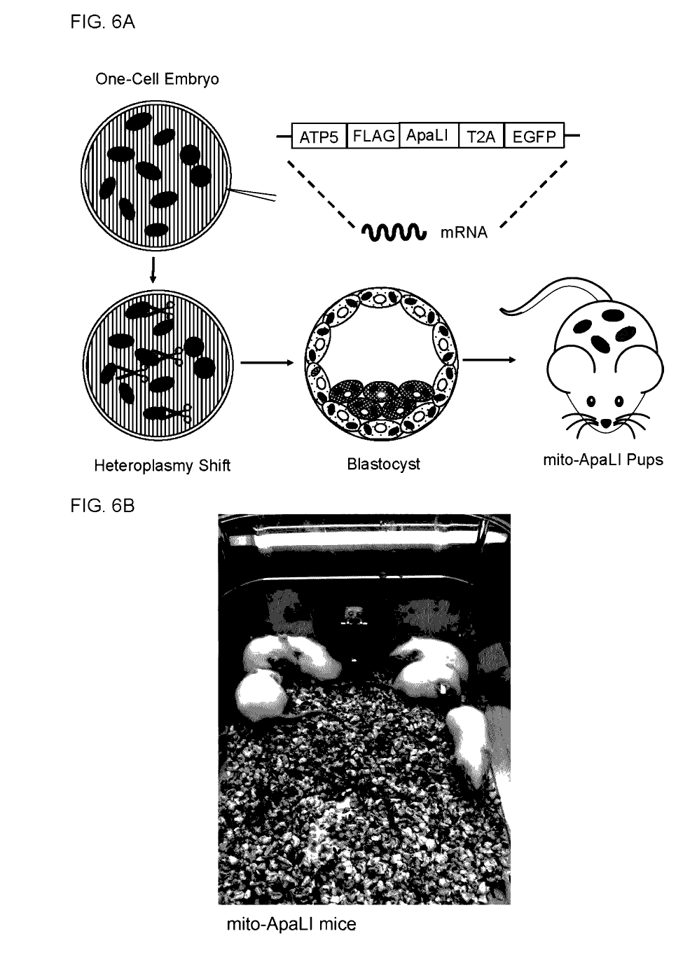

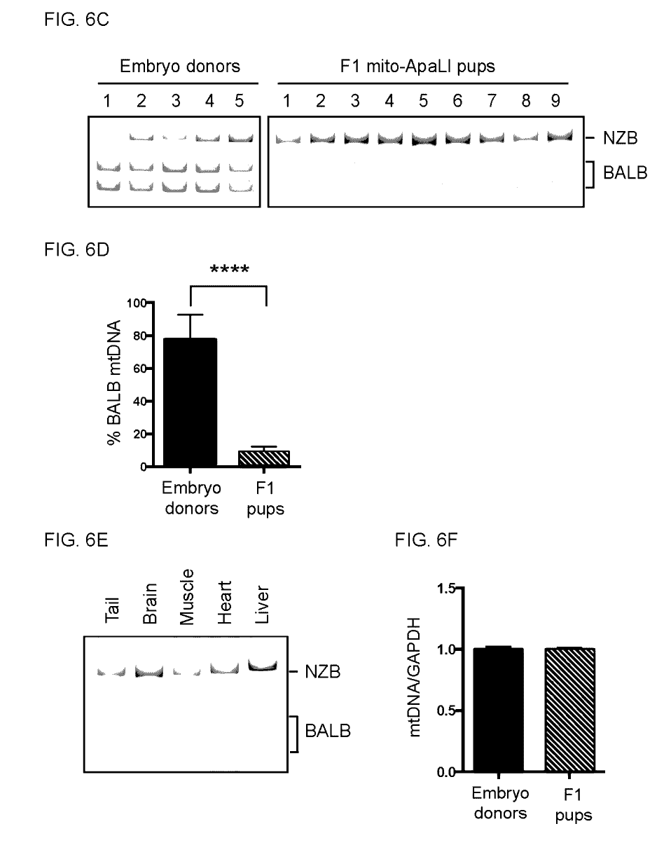

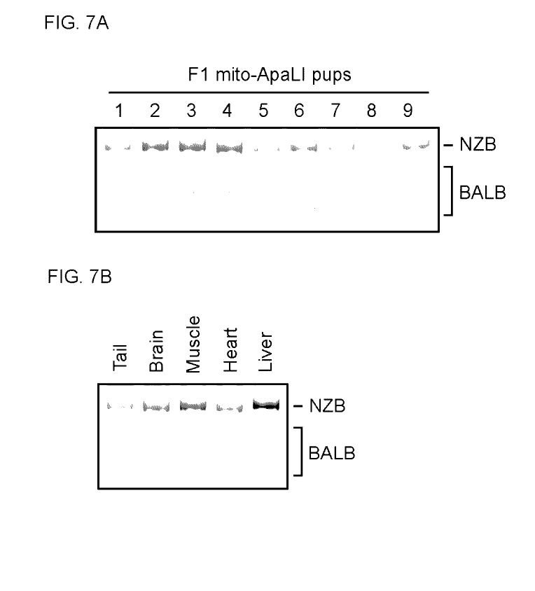

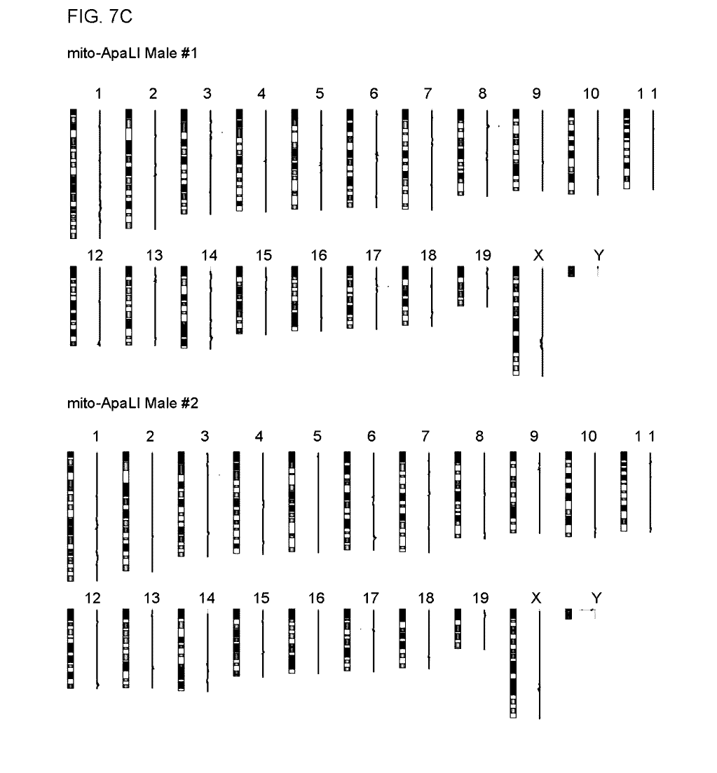

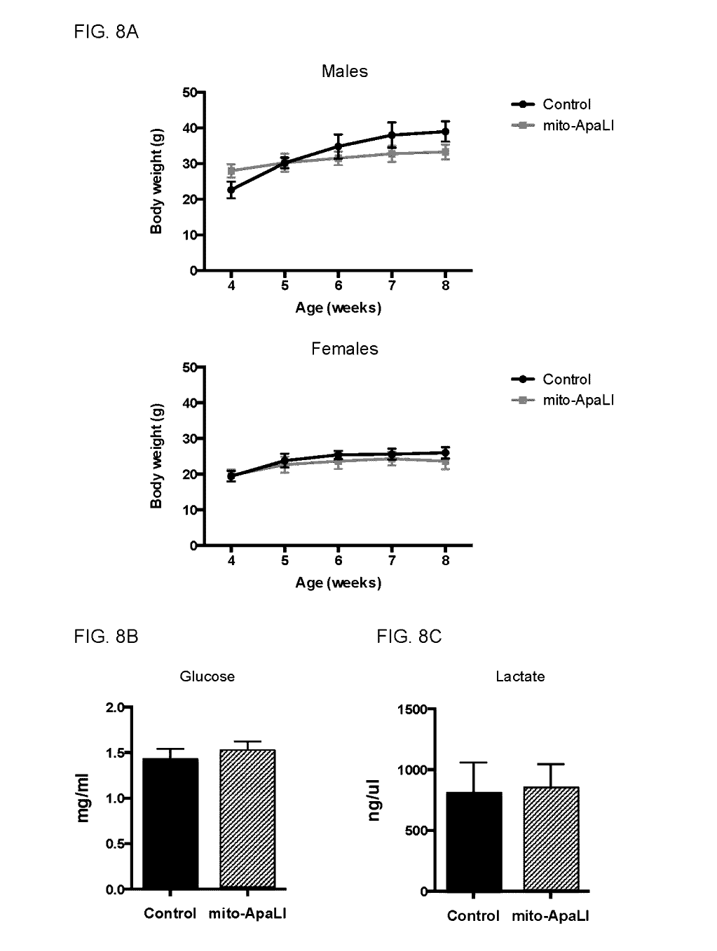

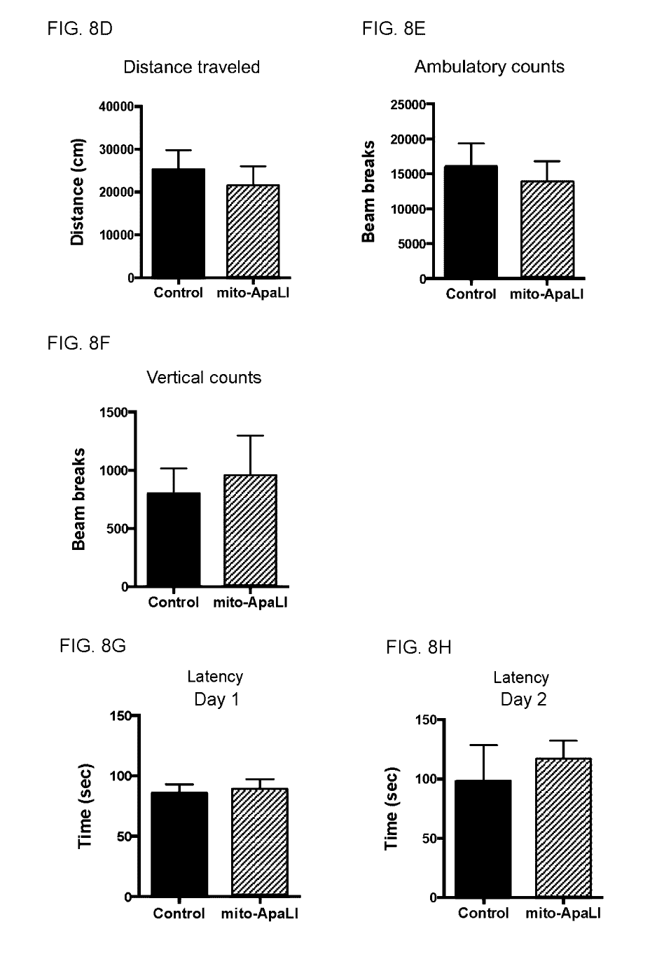

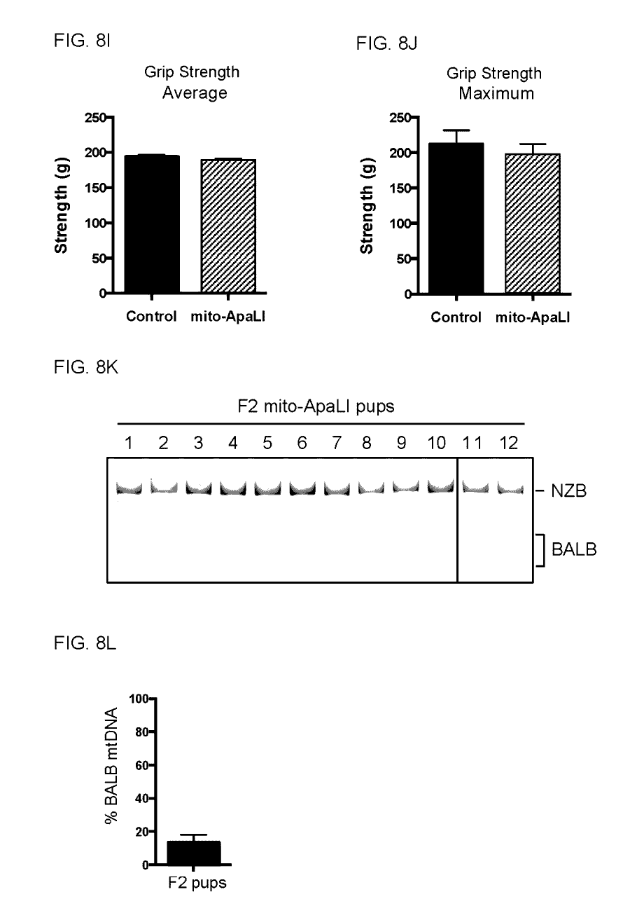

Next, it was investigated whether induction of mtDNA heteroplasmy shift could be utilized for preventing the transmission of mitochondrial diseases to the next generation. NZB/BALB one-cell embryos injected with mito-ApaLI mRNA were cultured in vitro until the blastocyst stage and transferred to pseudopregnant mice (see e.g., FIG. 6A). After a standard gestation period, pseudopregnant mice gave birth to live pups through natural delivery (see e.g., FIG. 6B). Most importantly, RFLP analysis of total DNA from F1 mito-ApaLI animals revealed a significant reduction of BALB mtDNA (see e.g., FIG. 6C). Further analysis demonstrated reduction of BALB mtDNA in the brain, muscle, heart, and liver. These data indicate the systemic clearance of a specific mtDNA in the offspring of heteroplasmic mothers (see e.g., FIG. 6D). Similarly, analysis at the HindIII region confirmed the specific reduction of BALB mtDNA in F1 mito-ApaLI animals (see e.g., FIG. 7A, FIG. 7B). Furthermore, analysis of mtDNA copy number showed normal mtDNA levels resulting from NZB mtDNA replication upon embryo implantation (see e.g., FIG. 6E). Comprehensive characterization of mito-ApaLI animals, both males and females, showed normal development, weight gain (see e.g., FIG. 8A), complete blood count (see e.g., TABLE 1) as well as normal blood levels of glucose and lactate, all potential indicators of mitochondrial dysfunction (Haas et al., 2007) (see e.g., FIG. 8B). Moreover, typical behavioral studies indicative of CNS defects (Ross et al., 2013), including open field, rotor-rod, grip strength, and sensory neuron screening, showed normal performance of mito-ApaLI animals (see e.g., FIG. 8C-8E).

TABLE 1
Blood analysis, related to FIG. 8. Analysis of mito-
ApaLI animals demonstrated normal complete blood count.

| Hematology | Normal Range | | mito-ApaLI mice | |
|---|---|---|---|---|
| White Wood cell count (K/μl) | 7.84 | (±2.25) | 6.18 | (±0.8) |
| Neutrophils (%) | 20.65 | (±4.74) | 15.24 | (±4.16) |
| Lymphocytes (%) | 74.71 | (±5.27) | 76.28 | (±5.7) |
| Monocytes (%) | 3.87 | (±1.54) | 7.73 | (±1.87) |
| Eosinophils (%) | 0.65 | (±0.34) | 0.56 | (±0.59) |
| Basophils (%) | 0.12 | (±0.08) | 0.16 | (±0.28) |
| Red blood cell count (M/μl) | 9.86 | (±0.59) | 10.03 | (±0.22) |
| Haemoglobin (g/dL) | 14.1 | (±1) | 15.01 | (±0.52) |
| HCT (%) | 45.5 | (±4.6) | 42.77 | (±1.76) |
| Mean Corpuscular volume (fL) | 46.1 | (±3.7) | 42.65 | (±1.65) |
| Mean Corpuscular | 14.3 | (±0.8) | 14.96 | (±0.58) |
| Haemoglobin (pg) | | | | |
| Mean Corpuscular | 31.1 | (±1.5) | 35.12 | (±0.75) |
| Haemoglobin | | | | |
| Concentration (g/dL) | | | | |
| RDW (%) | 18.1 | (±1.1) | 17.03 | (±0.43) |
| Platelets (K/μl) | 992 | (±145) | 838 | (±204) |
| MPV (fL) | 4.55 | (±0.46) | 4.67 | (±0.24) |

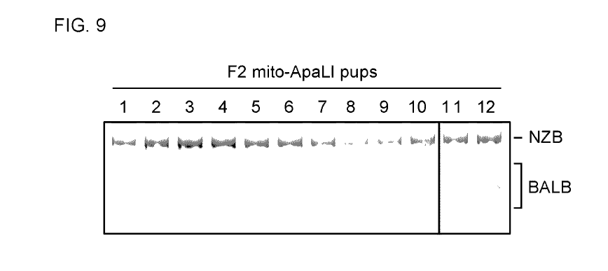

To assess potential off-target effects on the nuclear genome, comparative hybridization genomic (CHG) array and exome sequencing was performed. CGH array indicated normal genomic integrity of mito-ApaLI animals (see e.g., FIG. 7C). Confirming this result, exome sequencing demonstrated variant rates in ApaLI containing exomic regions comparable to non-ApaLI exomic regions, excluding the possibility of off-target effects of mito-ApaLI (0.0014 versus 0.0047 variants per hundred base pairs, respectively). Furthermore, mito-ApaLI animals were fertile, and RFLP analyses showed barely detectable levels of BALB mtDNA in the F2 generation (see e.g., FIG. 8F, FIG. 9). These results confirm the feasibility of mtDNA heteroplasmy shift to prevent the transgenerational transmission of mitochondrial diseases.

### Example 4

### Preventing the Transmission of Mitochondrial Genomes Using Mito-TALENS

The following examples describes the prevention of the transmission of mitochondrial genomes using mito-TALENs.

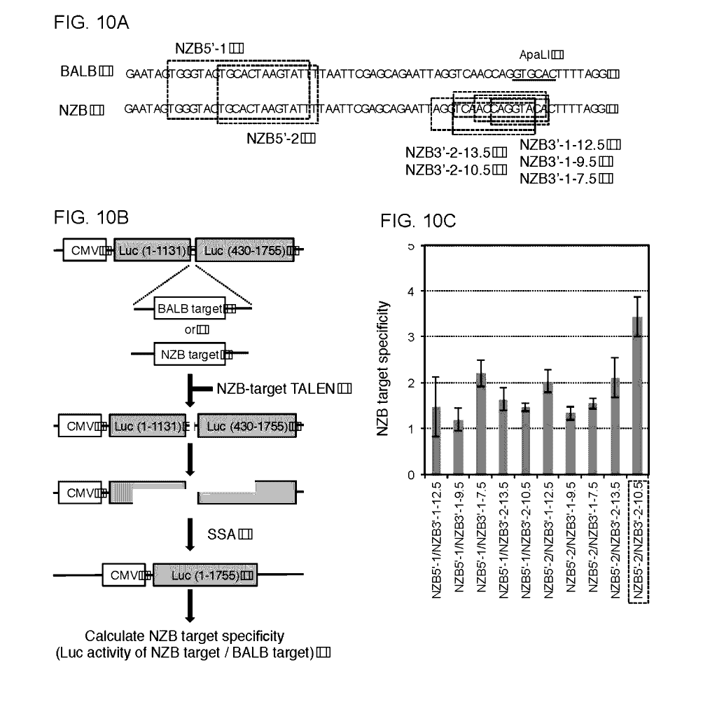

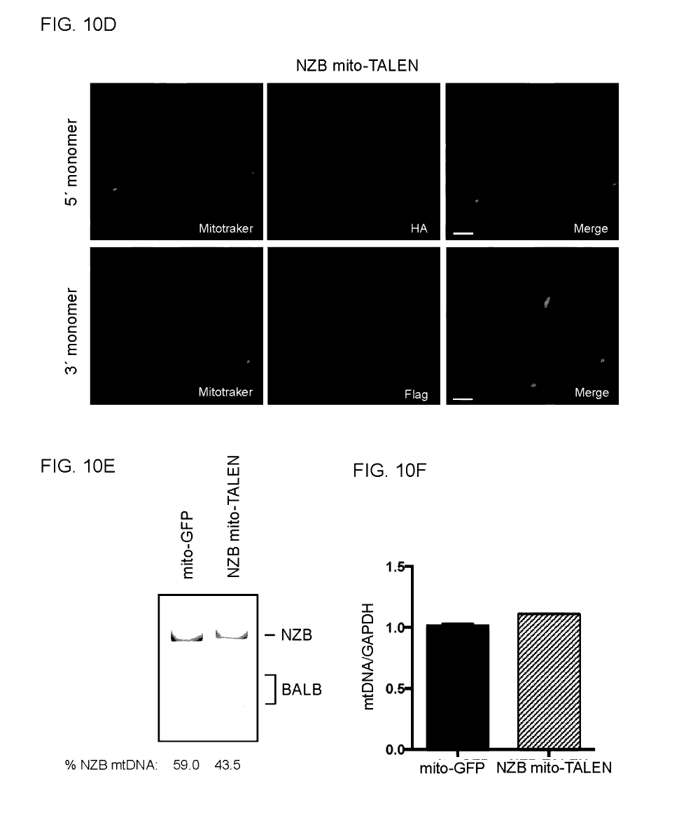

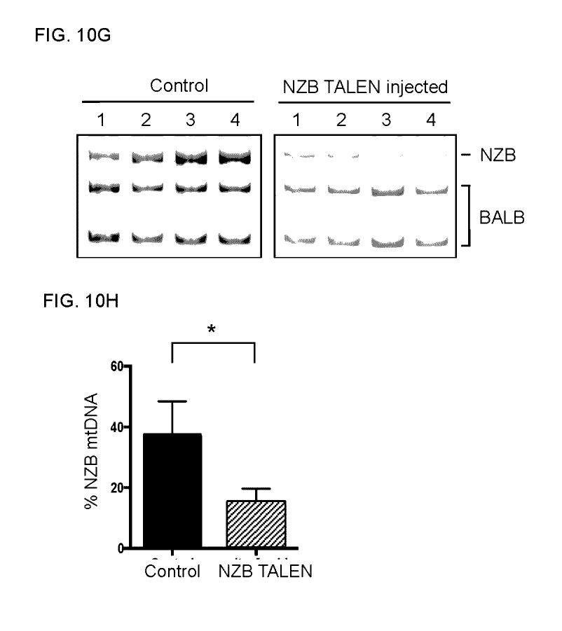

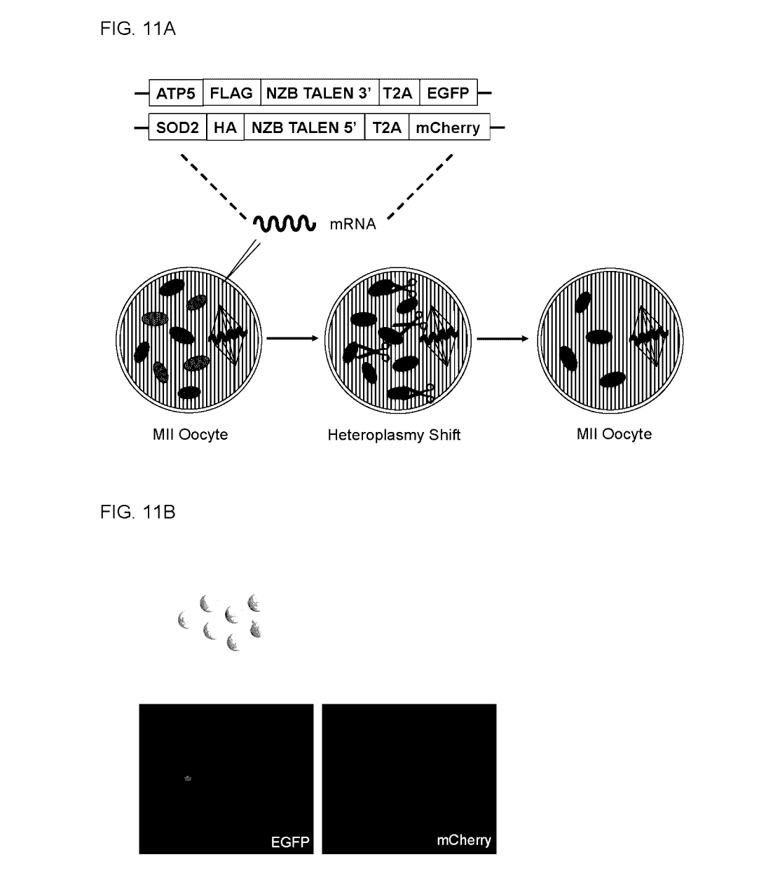

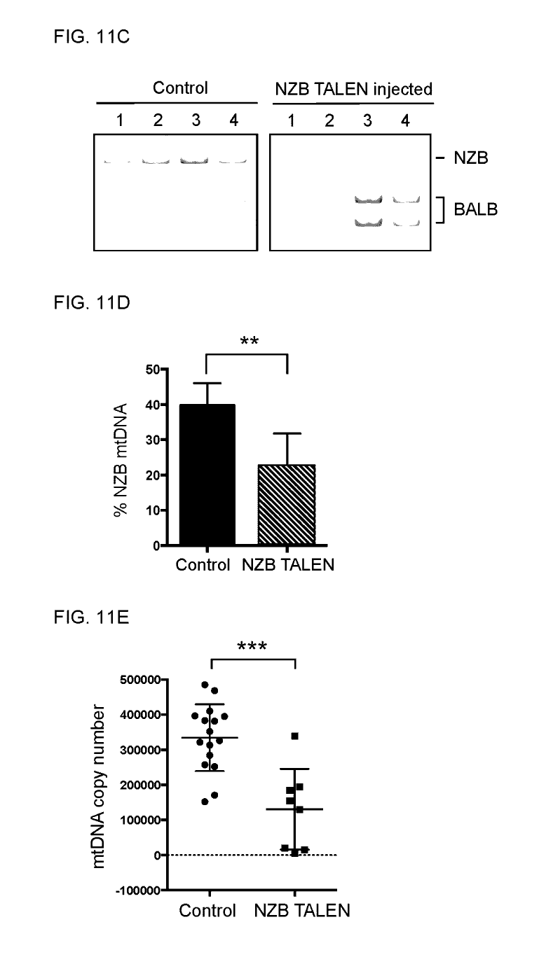

Despite the broad range of over 200 mtDNA mutations associated with mitochondrial diseases, only the human mutation m8993T>G are currently known to be responsible for two mitochondrial diseases: neurogenic muscle weakness, ataxia, and retinitis pigmentosa (NARP) and maternally inherited Leigh syndrome (MILS) generates a unique restriction site that can be targeted using the naturally occurring restriction endonuclease Xmal. For these reasons, alternative approaches to induce heteroplasmy shift based on the use of mitochondria-targeted transcription activator-like effector nucleases (TALENs) and zinc finger nucleases (ZFNs), which could be designed against virtually any mtDNA mutation or mutant mtDNA, have been recently developed by us and other groups (Bacman et al., 2013; Gammage et al., 2014; Minczuk et al., 2006; 2008). In order to evaluate the use of mito-TALENs to prevent the transmission of mitochondrial diseases, the specific elimination of NZB mtDNA in NZB/BALB oocytes was tested. For this purpose, a collection of TALENs against NZB mtDNA was generated and screened for a TALEN with the highest specificity against NZB mtDNA (see e.g., FIG. 10A-FIG. 10C). Under our design, the left monomer of the TALEN will bind to the common sequence of NZB and BALB mtDNA while the right monomer will preferentially recognize and bind to NZB mtDNA, dictating the specific cleavage of NZB mtDNA upon dimerization of the Fokl nuclease (see e.g., FIG. 10A). NZB TALEN monomers were targeted to mitochondria by the human ATP5b and SOD2 mitochondria targeting sequence and the ATP5b and SOD2 5′ and 3′ UTRs to promote co-translation import from mitochondrial associated ribosomes (Marc et al., 2002). In addition, an EGFP or mCherry reporter was also included in the constructs encoding each TALEN monomer (see e.g., FIG. 11A). Once again, the mitochondrial localization of the NZB TALEN was tested by immunostaining in NZB/BALB tail tip fibroblasts (TTFs) and observed robust co-localization of mitochondria-targeted NZB TALEN monomers (hereafter NZB mito-TALEN) with the mitochondrial dye Mitotracker (see e.g., FIG. 10D). Analysis of mtDNA by RFLP demonstrated induction of heteroplasmy shift in NZB/BALB cells by a specific reduction of NZB mtDNA after 72 hr in cells transfected with NZB mito-TALENs compared to control cells transfected with mito-GFP (see e.g., FIG. 10E). In addition, similar to mito-ApaLI, normal mtDNA copy number in NZB mito-TALEN transfected cells was found resulting from the replication of the remaining BALB mtDNA that compensated for the reduction of NZB mtDNA (see e.g., FIG. 10F).

Mito-TALENs was then tested for use in oocytes to specifically eliminate NZB mtDNA (see e.g., FIG. 11A). Fluorescent microscopy images revealed the expression of both NZB mito-TALEN monomers as indicated by EGFP and mCherry expression in oocytes (see e.g., FIG. 11B). RFLP analysis 48 hr after NZB mito-TALEN mRNA injection demonstrated the specific decrease of NZB mtDNA and a consequential increase in the relative BALB mtDNA levels (see e.g., FIG. 11C). RFLP analysis at the HindIII region confirmed the specific reduction of NZB mtDNA upon injection of NZB mito-TALEN in NZB/BALB MII oocytes (see e.g., FIG. 10G). Analysis of mtDNA copy number by qPCR revealed a decrease in mtDNA copy number following NZB mito-TALEN injection in oocytes in agreement with the lack of mtDNA replication in oocytes (see e.g., FIG. 11D). These results demonstrate the potential of custom designed mito-TALENs for the specific elimination of mitochondrial genomes in the germline aimed at preventing the transmission of mitochondrial diseases.

### Example 5

### Specific Reduction of Human Mutated Mitochondrial Genomes

The following example describes the specific reduction of human mutated mitochondrial genomes responsible for mitochondrial diseases in mammalian oocytes.

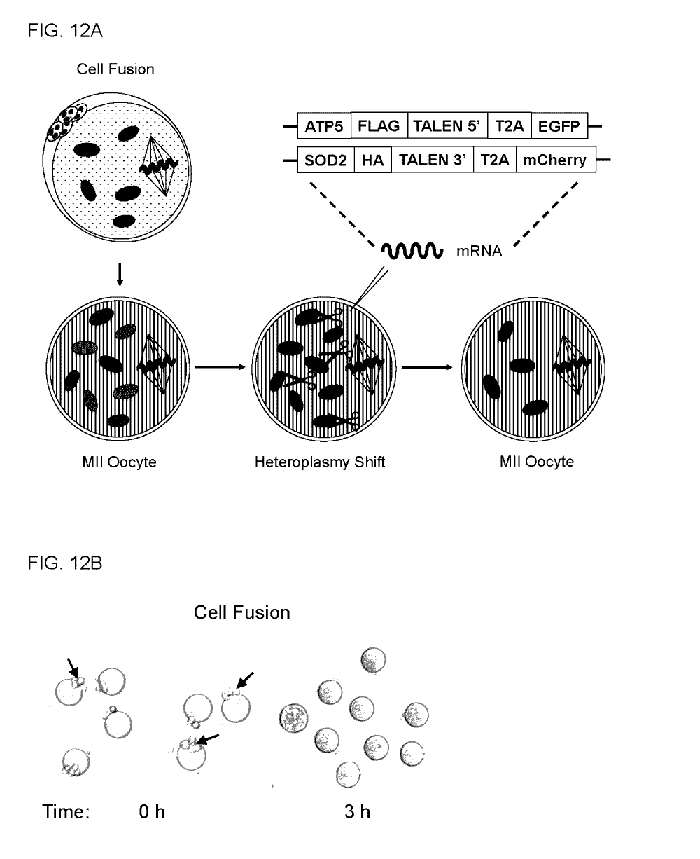

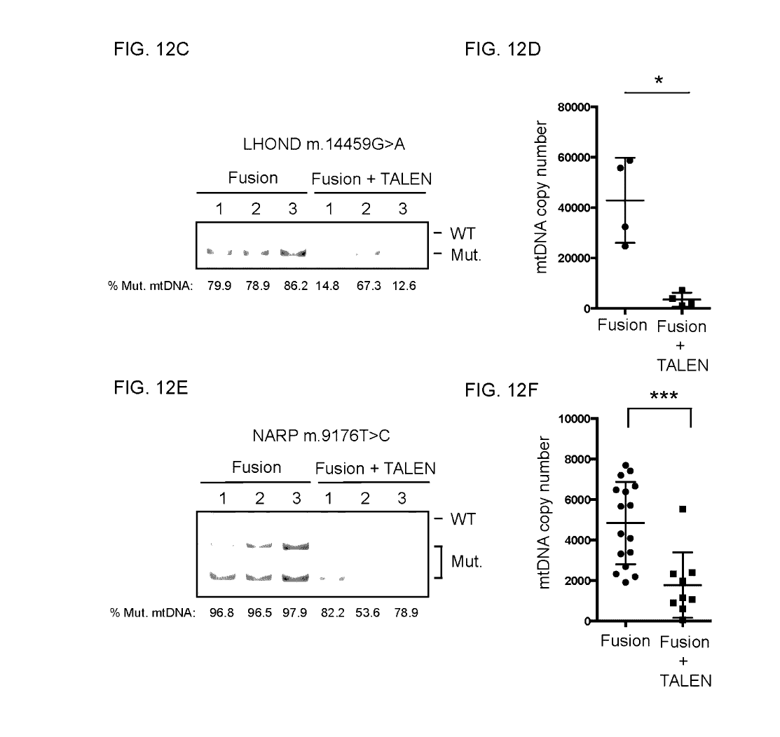

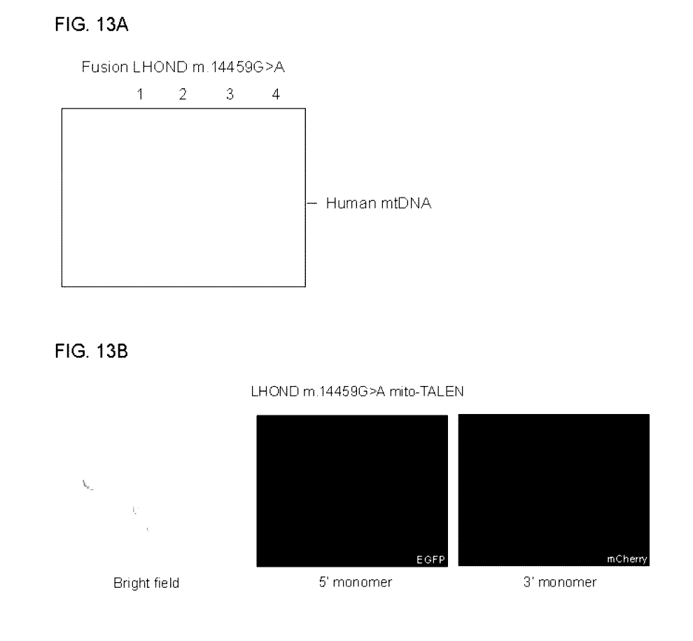

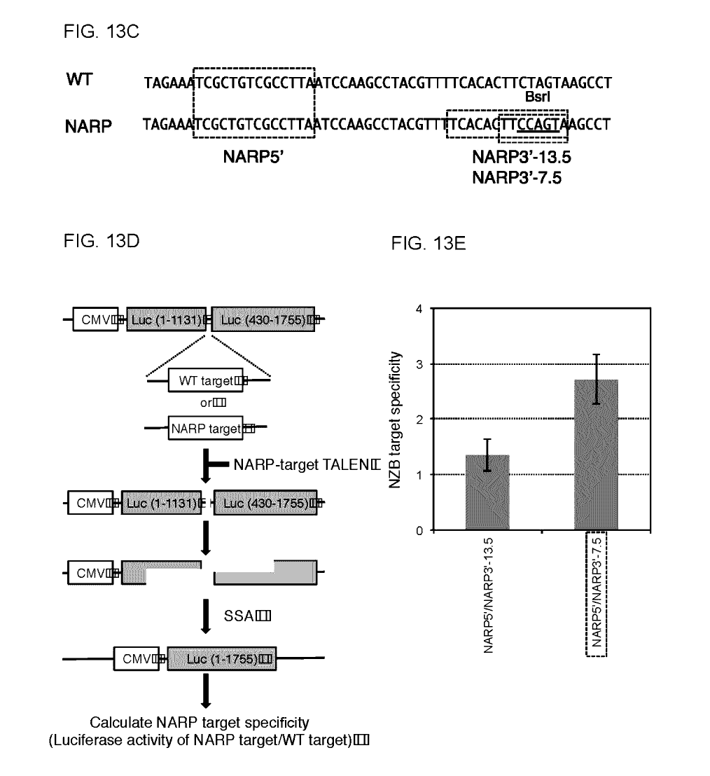

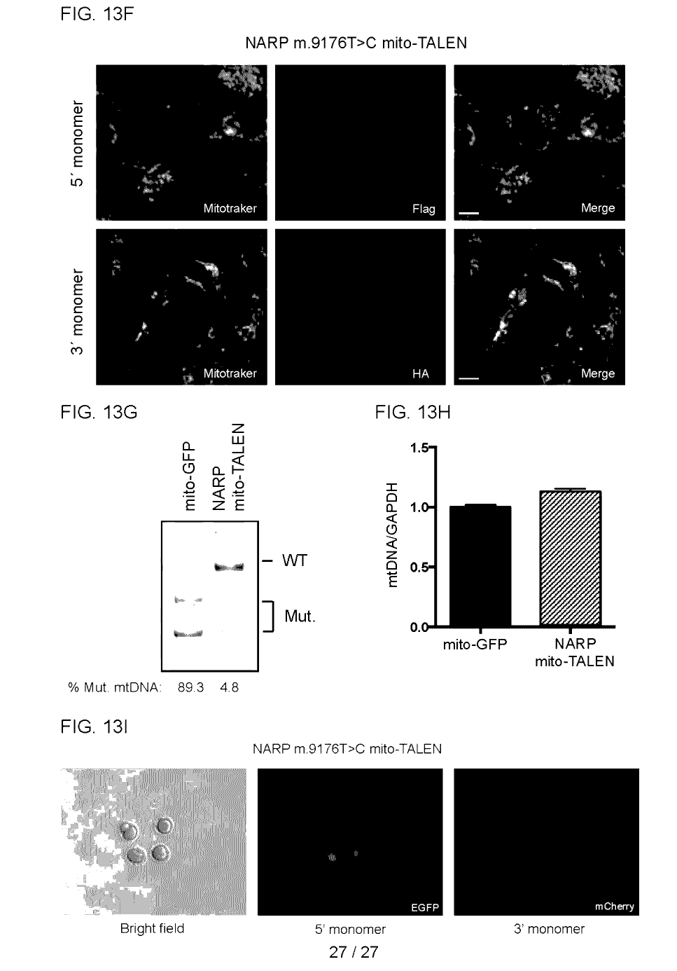

In order to evaluate the approach to prevent the transmission of human mitochondrial diseases we tested the use of mitochondria-targeted nucleases against mutated mitochondrial genomes responsible for two mitochondrial diseases: Leber's hereditary optic neuropathy and dystonia (LHOND) and NARP (Jun et al., 1994; Taylor and Turnbull, 2005). Due to the limited number of available patients and the difficulty in obtaining oocytes from these patients, we generated artificial mammalian oocytes carrying mutated genomes by cellular fusion of patient cells and mouse oocytes using Sendai virus (see e.g., FIG. 12A). Although this model has limitations compared to patient oocytes, it allowed for testing the potential of the methodology for the specific elimination of pathogenic human mtDNAs in mammalian oocytes. For this purpose, we first tested the fusion of 143B osteosarcoma cybrid cells harboring the LHOND m.14459G>A mutation to mouse MII oocytes (see e.g., FIG. 12B). After 3 hr, complete fusion was observed and no individual cells were detected under the zona pellucida of oocytes (see e.g., FIG. 12B). LHOND-fused oocytes were incubated for 48 hr and collected for analysis. PCR analysis using primers specific against the human mtDNA region containing the LHOND m.14459G>A mutation allowed for the detection of LHOND mtDNA in fused oocytes (see e.g., FIG. 13A). Next, we tested whether the LHOND mito-TALEN that we have recently reported could be used for the specific elimination of LHOND mtDNA in oocytes (Bacman et al., 2013). For this purpose, MII oocytes harboring LHOND mtDNA were injected with mRNA encoding the LHOND mito-TALEN 3 hr after cell fusion. Fluorescent microscopy images revealed the expression of both LHOND mito-TALEN monomers as indicated by EGFP and mCherry expression (see e.g., FIG. 13B). RFLP analysis 48 hr after mRNA injection demonstrated the specific reduction of LHOND mtDNA in fused oocytes (see e.g., FIG. 12C). Analysis of mtDNA copy number by qPCR confirmed a significant reduction of human mutated LHOND mtDNA upon injection of LHOND mito-TALENs in fused oocytes (see e.g., FIG. 12D). Finally, to demonstrate the potential of this approach against other mitochondrial diseases we decided to used a similar strategy to test the elimination of human mitochondrial genomes carrying the mutation NARP m.9176T>C. For this purpose, we first generated a collection of TALENs against NARP mtDNA and screened for a TALEN with the highest specificity against the mutation NARP m.9176T>C (see e.g., FIG. 13C-FIG. 13E). NARP mito-TALEN monomers were targeted to mitochondria by the ATP5b and SOD2 mitochondria targeting sequence and the ATP5b and SOD2 50 and 30 UTRs (see e.g., FIG. 12A). Immunostaining in NARP patient cells revealed a robust co-localization of mitochondria-targeted NARP mito-TALEN monomers with the mitochondrial dye Mitotracker (see e.g., FIG. 13F). Subsequently, we tested the induction of heteroplasmy shift by NARP mito-TALEN using immortalized NARP patient cells. Analysis of mtDNA by RFLP demonstrated induction of heteroplasmy shift in NARP cells with a reduction in NARP mtDNA after 72 hr in cells transfected with the NARP mito-TALEN compared to cells transfected with mito-GFP (see e.g., FIG. 13G). In addition, we found normal mtDNA copy numbers in NARP mito-TALEN transfected cells resulting from the replication of the remaining mtDNA (see e.g., FIG. 13H).

Next, similar to LHOND, we tested the specific elimination of NARP mitochondrial genomes in oocytes. As before, patient cells harboring the NARP m.9176T>C mutation were fused to MII oocytes using Sendai virus and injected with NARP mito-TALEN 3 hr after fusion. Fluorescent reporters for both NARP mito-TALEN monomers were observed in oocytes as indicated by EGFP and mCherry expression (see e.g., FIG. 13I). RFLP analysis 48 hr after mRNA injection demonstrated the specific reduction of NARP mtDNA in fused oocytes (see e.g., FIG. 12E). Analysis of mtDNA copy number by qPCR confirmed a significant reduction of human mutated NARP mtDNA upon injection of NARP mito-TALENs in fused oocytes (see e.g., FIG. 12F). We speculate that the low levels of wild-type mtDNA carried by the NARP patient cells, together with the lack of mtDNA replication in oocytes, might be the reason why we failed to detect a significant increase in wild-type human mtDNA upon NARP mito-TALEN injection. Collectively, these results show the potential for a custom-designed mito-TALENs for the specific elimination of clinically relevant mutated mitochondrial genomes responsible for human mitochondrial diseases in the germline.

### Example 6

### Summary of Examples 1-5

In summary, novel strategies for preventing germline transmission of mitochondrial diseases through the induction of mtDNA heteroplasmy shift in oocytes and embryos are described in the above examples. A heteroplasmic mouse model carrying two different mtDNA haplotypes: NZB and BALB was used.

First, it was demonstrated that injection of mRNA encoding mitochondria-targeted ApaLI restriction enzyme into oocytes, as well as into one-cell embryos, led to the generation of live animals with significantly reduced levels of the BALB mtDNA haplotype. These animals displayed normal behavior, development, gross genomic integrity and fertility. Moreover, their progeny (F2 generation) maintained significantly reduced levels of BALB mtDNA. These results demonstrate the potential of germline heteroplasmy shift to prevent the transgenerational transmission of mitochondrial genomes.

In addition, injection of mRNA encoding mitochondria-targeted NZB mito-TALEN into oocytes led to a significant reduction of NZB mtDNA levels. Finally, fusion of human patient cells carrying mtDNA mutations to mouse oocytes followed by injection of mito-TALENs against the human mtDNA mutations demonstrated a specific reduction in the levels of mutated mtDNA.

The use of restriction nucleases for the induction of heteroplasmy shift has been previously demonstrated in the NZB/BALB mouse as well as in patient somatic cells by us and other groups (Alexeyev et al., 2008; Bacman et al., 2010; 2012). However, the application of restriction enzymes to target clinically relevant mutations is limited to only m8993T>G, which is responsible for some cases of NARP and MILS, a mutation that generates a unique restriction site that can be targeted using the restriction endonuclease Xmal (Alexeyev et al., 2008). The use of other approaches using different types of nucleases including TALENs might allow for the custom-designed targeting of a wider range of human mitochondrial mutations responsible for mitochondrial diseases. Along this line, several reports have recently demonstrated the use of mitochondria-targeted TALENs and zinc finger nucleases (ZFNs) for the specific elimination of mutated mitochondrial genomes in somatic cells (Bacman et al., 2013; Gammage et al., 2014; Minczuk et al., 2006; 2008). Although, when compared to mitochondria-targeted restriction endonucleases, the use of mito-TALENs for preventing transmission of mitochondrial diseases in the germline may be less robust. However, it is presently thought that their therapeutic use will achieve specific reduction of mutated mitochondrial genomes below the threshold levels (60%-95%) currently believed to be required for biochemical and clinical defects to manifest (Russell and Turnbull, 2014). In addition, it is presently thought that the future development and application of more specific and efficient gene editing technologies will allow for a higher significant reduction of mutated mtDNA levels in the germline.

Transmission of mitochondrial diseases by female carriers directly correlates with the levels of mutated mtDNA present in oocytes. In many cases, asymptomatic female carriers with intermediate levels of mutant load may produce oocytes with different ranges of mutated mtDNA (Chinnery et al., 2000; Cree et al., 2009). Due to the lack of mtDNA replication in oocytes and pre-implantation embryos, targeting of mutated mtDNA in oocytes with high mutant loads using the approach presented here may lead to a dramatic reduction in mtDNA copy number. In mice, embryos with mitochondria levels below a specific threshold develop normally during the pre-implantation stages but subsequently fail to implant in the uterus or undergo development arrest (Wai et al., 2010). Consequently, oocytes containing high levels of mutated mtDNA that are subjected to heteroplasmy shift may result in embryos with low mtDNA copy number that may fail to implant in the uterine wall. In this case, though heteroplasmy shift may not result in a viable embryo, it would attain the goal of hampering the development and implantation of embryos with high mutant loads, thereby preventing the transmission of mitochondrial diseases to the next generation. In this scenario, pre-implantation genetic diagnosis (PGD) could be used as a complementary approach to select embryos with mtDNA copy numbers sufficient for implantation.

Due to the non-Mendelian segregation of mtDNA, current therapeutic approaches, including genetic counseling and pre-implantation genetic diagnosis (PGD), can only partially reduce, but not eliminate, the risk of transmission of mitochondrial diseases (Brown et al., 2006). The recent development of mitochondrial replacement techniques based on spindle, pronuclear, or polar body transfer into healthy enucleated donor oocytes or embryos, currently under review by US and UK regulatory agencies, represent a valid and powerful alternative to current approaches (Craven et al., 2010; Paull et al., 2013; Tachibana et al., 2013; Wang et al., 2014). Mitochondrial replacement techniques involve a series of complex technical manipulations of nuclear genome between patient and donor oocytes that can result in the generation of embryos carrying genetic material from three different origins. For these reasons, mitochondrial replacement techniques have raised some biological, medical, and ethical concerns (Hayden, 2013; Reinhardt et al., 2013). Despite their great potential, more studies are still required to show that these techniques are safe in human oocytes. The approach presented in the above examples relies on a single injection of mRNA into patient oocytes, which is technically simpler and less traumatic to the oocyte compared to mitochondrial replacement techniques (Craven et al., 2010; Paull et al., 2013; Tachibana et al., 2013; Wang et al., 2014). Importantly, the technique described herein does not require healthy donor oocytes, thus avoiding ethical issues related to the presence of donor mtDNA.

Induction of mtDNA heteroplasmy shift using restriction endonucleases or TALENs has shown potential to eliminate mutated mitochondrial genomes in the germline, and consequently, prevent the transgenerational transmission of mitochondrial diseases. In addition, because mtDNA mutations in the germline have been recently linked to aging (Ross et al., 2013), this strategy could also be applied to prevent the transmission of mtDNA variants with potential roles in aggravating aspects of human aging and age-associated diseases.

### Example 7

### Experimental Procedures

The following example describes the experimental procedures used in Examples 1-6.

### Plasmids.

A synthetic gene coding for the ApaLI restriction endonuclease with a C-terminal HA (Hemagglutinin antigen) tag was purchased from Integrated DNA Technologies (Coralville) with codon usage optimized for mammalian translation. For the generation of the mito-ApaLI construct, ApaLI was subcloned into the pVAX plasmid containing the mitochondria localization signal derived from ATP5b, a unique Flag immunotag in the N terminus, 50 and 30 UTR from ATP5b to localize the mRNA to ribosomes associated with mitochondria, an independent fluorescent marker to select for expression (enhance GFP [EGFP]) and a recoded picornaviral 2A-like sequence (T2A′) between the mito-ApaLI and the fluorescent marker. Subsequently, the fragment described was subcloned into the pcDNA3 plasmid containing a T7 promoter for in vitro transcription. For the generation of the mito-GFP construct, EGFP was subcloned into the previously described pVAX construct lacking the independent fluorescent marker and the recoded picornaviral 2A-like sequence (T2A′) but containing a T7 promoter. For the generation of ApaLI construct, ApaLI RE was subcloned into the previously described pVAX plasmid lacking the N terminus mitochondria localization signal derived from ATP5b and the 5′ and 3′ UTR from ATP5b with a T7 promoter. Cloning was done using the using the In-Fusion HD cloning kit (Clontech Laboratories).

### Construction of Mito-TALENs.

TALEN target sites for NZB and NARP m.9176T>C were identified using the TAL effector-Nucleotide Targeter (TALE-NT) software (Christian et al., 2010). To increase TALEN specificity, TALEN with targeting sequences of various lengths ranging from 7.5 to 13.5 base pairs were designed. TALENs were constructed into the TALEN cloning vector of the TALE Toolbox kit from Addgene (cat#1000000019) (Sanjana et al., 2012), and the TALENs recognizing the target sites were constructed using the Golden Gate Assembly method.

Mito-TALEN, were constructed by addition of mitochondria localization signals derived from ATP5b or SOD2 mitochondria localization signal, inclusion of a unique immuno-tag in the N terminus of the mature protein (hemagglutinin [HA] or Flag), inclusion of a 3′ UTR from a mitochondrial gene known to localize mRNA to ribosomes on mitochondria, inclusion of an independent fluorescent marker to select for expression (EGFP in one monomer and mCherry in the other) and inclusion of a recoded picornaviral 2A-like sequence (T2A′) between the mitoTALEN and the fluorescent marker.

### Animals.

All animal procedures were performed according to NIH guidelines and approved by the Committee on Animal Care at Salk Institute. NZB/BALB heteroplasmic founder females were originally generated (Jenuth et al., 1996). NZB/BALB colony was maintained by breeding the females with BALB/cByJ males. Tail tip genotyping was routinely performed in order to exclude females carrying low levels of one of the two mtDNA haplotypes. BALB/c, BALB/cByJ and NZB mice were obtained from Jackson laboratory.

### Cells, Transfection, and Sorting.

Simian virus 40 (SV40) immortalized NZB/BALB fibroblasts containing NZB and BALB mtDNA were derived from tail tip of NZB/BALB mice. Human patient cells harboring the NARP m.9176T>C mutation were obtained by skin biopsy after signed informed consent of the donor and with the approval of the Institutional Review Board of the Hospital Clinic, Spain. Cells were immortalized using SV40 and cultured at 37° C. in DMEM (Invitrogen) containing GlutaMAX, non-essential amino acids and 10% fetal bovine serum (FBS). 143B osteosarcoma cybrid cells harboring the LHOND m.14459G>A mutation were obtained and cultured as previously described (Bacman et al., 2013). Cells were transfected with Lipofectamine 2000 (Invitrogen) according to the manufacturer's instructions. After 72 hr, cells were sorted using a BD Influx (Becton, Dickinson and Company) by gating on single-cell fluorescence using a 488-nm laser with a 505LP, 530/40 filter set for EGFP and a 561-nm laser with a 600LP, 610/20 filter set for mCherry. Total DNA was extracted from sorted cells using the DNeasy Blood and Tissue Kit (QIAGEN) following the protocol suggested by the manufacturer.

### Single Strand Annealing Reporter Assay.

The pGL4-SSA reporter construct was purchased from Addgene (#42962). The construction of TALEN target sequence followed previous reports (Targeted mutagenesis in the sea urchin embryo using zinc-finger nucleases). NZB target sequence is amplified from mitochondria DNA of NZB/BALB cells using the following primers (ApaL_luc-F (SEQ ID NO: 1): TGGACTAGGGTCTCGCGGGAATAGTGGGTACTGC and ApaL_luc-R (SEQ ID NO: 2): CGCTCCTAGGTCTCATTATAACAAAAGCATGGGCAGTTACG). The PCR products were subcloned into Bsal site of pGL4-SSA and NZB target reporter (pGL4SSA-NZB) plasmids were selected by sequencing. WT and NARP target mtDNA sequences were amplified from healthy donor or NARP patient cells with the following primers (NAPR_luc-F (SEQ ID NO: 3): TGGACTAGGGTCTCTATCCTAGAAATCGC TGTCGCC and NAPR_luc-R (SEQ ID NO: 4): CGCTCCTAGGTCTCATAGGCATGTGATTGGT GGGTC). The PCR products were subcloned into Bsal site of pGL4-SSA, and constructed WT (pGL4SSA-WT) and NARP target reporter (pGL4SSA-NARP) plasmids. To detect TALEN activity for the target site, SSA assay was done with SSA reporter plasmids (pGL4SSA-NZB, pGL4SSA-WT or pGL4SSA-NARP) following previous reports (Targeted mutagenesis in the sea urchin embryo using zinc-finger nucleases). To determine TALEN specificity for the NZB or NARP target site, the detected luciferase activities for the target sites (i.e., NZB or NARP) were normalized to the non-target sites (i.e., WT).

Production of mRNA.

In vitro transcription of mRNA was performed using mMESSAGE mMACHINE T7 ULTRA kit (Life Technologies) according to the manufacturer's instructions using linearized and gel purified (QIAGEN) plasmid template. The mRNA was purified using MEGAclear kit (Life Technologies) and quantified using Nano-Drop 8000 (Thermo Scientific).

Oocyte Collection and mRNA Injection.

Female mice were superovulated with pregnant mares serum gonadotropin (PMSG) and human chorionic gonadotropin (hCG). MII oocytes were collected 14 hr after hCG injection in M2 medium (Millipore) and freed of cumulus cells using hyaluronidase. For collection of 1-cell embryos, superovulated female mice were mated to BALB/c males and fertilized embryos were collected 18-20 hr after hCG injection from oviduct. mRNA (50-250 ng/ml) was injected into the cytoplasm of MII oocytes and fertilized embryos in M2 medium using Eppendorf micromanipulator. The injected MII oocytes were in vitro cultured in KSOM (Millipore) for 48 hr before analysis. The injected embryos were cultured in KSOM at 37° C. under 5% CO₂ in air until blastocyst stage. Subsequently, blastocysts were collected for analysis or transferred to BALB/c pseudopregnant females. Live pups were obtained by natural delivery.

### Cell Fusion.

Cell fusion was achieved by using inactivated Sendai virus (GenomeOne, Cosmo Bio). Sendai virus stock solutions were prepared according to the manufacturer instructions and further diluted 1:20 in cell fusion buffer. The 143B osteosarcoma cybrid cells harboring LHON m.14459G>A mutation and patient cells harboring NARP m9176T>C mutation were used for fusion with mature MII oocytes. Cells were cultured for 48 hr in DMEM no glucose medium supplemented with galactose before using for cell fusion to increase mtDNA content. On the day of fusion, cells were trypsinized and resuspended in M2 medium. For each MII oocyte, five cells briefly placed in Sendai virus were injected under the zona pellucida. After 3 hr successfully fused oocytes were selected for mito-TALEN mRNA injection. Lastly, surviving oocytes were cultured in KSOM for 48 hr before analysis.

### Immunofluorescence.

Cells were seeded on coverslips before transfection. Forty-eight hours after of transfection cells were incubated in the presence of 350 nM Mitotracker (Invitrogen) for 30 min. Subsequently, cells were fixed and permeabilized with 4% PFA and 0.1% Triton X-100, respectively. After fixation, cells were blocked for 1 hr at room temperature with 1% BSA/PBS. Next, cells were incubated with an anti-Flag M2 primary antibody (Sigma) or anti-HA antibody (Millipore) overnight at 4° C. The next day, cells were washed three times with PBS and incubated for 1 hr at room temperature with Alexa Fluor 488-conjugated donkey antibodies to goat IgG (Molecular Probes) or Alexa Fluor 647-conjugated donkey antibodies to mouse IgG and 10 min with Hoechst 33342 (0.5 mg ml⁻1 in PBS) (Invitrogen). Finally, cells were washed three times with PBS and mounted using Fluoromount-G (Southernbiotech). Confocal image acquisition was performed using a Zeiss LSM 780 laser-scanning microscope (Carl Zeiss Jena).

### “Last-Cycle Hot” PCR and RFLP.

Total DNA from cells, tail biopsies, and oocytes/embryos were used to determine mtDNA heteroplasmy by “Last-cycle hot” PCR using the mtDNA 50 Fluorescein amidite (FAM) labeled primers as listed in TABLE 2. NZB/BALB PCR products were digested with ApaLI or HindIII, which digests BALB mtDNA at positions 5461 (ApaLI targeting site) and 9136 respectively. NARP PCR products were digested with BsrI which digest mutated NARP mtDNA at position 9176. The levels of LHON m.14459G>A were determined as previously reported (Bacman et al., 2013). Digested PCR products were subjected to electrophoresis in an 12% polyacrylamide gel. The fluorescein signal was quantified using a Typhoon 8600 system (Molecular Dynamics) and gels were quantified using ImageQuant 5.2 (Molecular Dynamics). Quantification of mtDNA Copy Number Absolute mtDNA copy numbers were quantified by real-time PCR using iQSyber Green on Bio-Rad iCycler (Bio-Rad). Individual oocytes and embryos were transferred into lysis buffer (200 mM KOH) and incubated for 10 min at 65° C. The reaction was neutralized by addition of 200 mM HCl. Absolute mtDNA copy number per 1 ml of lysate was calculated using a standard curve derived from the Q-PCR amplification of a fragment of mtDNA genome. Briefly, primers listed in TABLE 2 were used to quantify the absolute level of mtDNA in samples. First, a standard curve was generated by a 10-fold serial dilution of a PCR product obtained using Standard curve primers for the different regions of mtDNA analyzed. Subsequently, to quantify the absolute level of mtDNA, quantitative real-time PCR was performed using qPCR primers listed in TABLE 2.

TABLE 2
Primer Sets Used in This Study, Related to Experimental Procedures Section. Primer
sets used for Last-cycle hot′ PCR and quantification of mtDNA copy number.
′Last-cycle hot′ PCR primers*

| | Restriction |
|---|---|
| Primer Sequence (5′-3′) | enzyme |
| NZB/BALB F_5184 GGCGGTAGAAGTCTTAGT (SEQ ID NO: 5) | ApaLI |
| R_5646 GGAGAAGGAGAAATGATGG (SEQ ID NO: 6) | |
| NZB/BALB F_8811 GGCCACCACACTCCTATTGT (SEQ ID NO: 7) | HindIII |
| R_9305 ATGCTGCGGCTTCAAATCCG (SEQ ID NO: 8) | |
| LHON F_14425 CCCCCATGCCTCAGGATACTCCTCAATAGTGATC (SEQ ID NO: 9) | BclI |
| R_14552 TGATTGTTAGCGGTGTGGTCGGGTGTGT (SEQ ID NO: 10) | |
| NARP F_8904 CCACTTCTTACCACAAGGCACACCTACACC (SEQ ID NO: 11) | BsrI |
| R_9319 AGGCCTAGTATGAGGAGCGTTATGGAGT (SEQ ID NO: 12) | |

*5′ Fluorescein amidite (FAM) labeled. Mismatch codons are marked in bold.

| Primers for quantification of mtDNA copy number | |
|---|---|
| | Restriction |
| Primer Sequence (5′-3′) | enzyme |
| NZB/BALB F_5184 GGCGGTAGAAGTCTTAGT (SEQ ID NO: 13) | Standard curve |
| R_5646 GGAGAAGGAGAAATGATGG (SEQ ID NO: 14) | (ApaLl site) |
| NZB/BALB F GAGCGGGAATAGTGGGTACTG (SEQ ID NO: 15) | qPCR- |
| R ACAAAAGCATGGGCAGTTACG (SEQ ID NO: 16) | ApaLI site |
| NZB/BALB F_8811 GGCCACCACACTCCTATTGT (SEQ ID NO: 17) | Standard curve |
| R_9305 ATGCTGCGGCTTCAAATCCG (SEQ ID NO: 18) | (HindIII site) |
| NZB/BALB F CAAGCCCTACTAATTACCATTATAC (SEQ ID NO: 19) | qPCR- |
| R AGTCCATGGAATCCAGTAGCC (SEQ ID NO: 20) | HindIII site |
| LHON F_14425 CCCCCGAGCAATCTCAATTA (SEQ ID NO: 21) | Standard curve |
| R_14552 TGATTGTTAGCGGTGTGGTCGGGTGTGT (SEQ ID NO: 22) | |
| LHON F CCCCCATGCCTCAGGATACTCCTCAATAG (SEQ ID NO: 23) | qPCR |
| R TGATTGTTAGCGGTGTGGTCGGGTGTGT (SEQ ID NO: 24) | |
| NARP F_8904 CCACTTCTTACCACAAGGCACACCTACACC (SEQ ID NO: 25) | Standard curve |
| R_9319 AGGCCTAGTATGAGGAGCGTTATGGAGT (SEQ ID NO: 26) | |
| NARP F CTGACTATCCTAGAAATCGC (SEQ ID NO: 27) | qPCR |
| R GATTGGTGGGTCATTATGTG (SEQ ID NO: 28) | |

### Blood and Plasma Parameters.

Blood collection was performed by sub-mandibular bleeding. Whole EDTA blood samples were analyzed in duplicates for Complete Blood Count (CBC) on a Hemavet 950FS Multi Species Hematology System (Drew Scientific). For glucose and lactate analysis was determined using the Glucose (GO) Assay Kit (Sigma) according to the manufacturer's instructions. Plasma lactate concentration was determined using the Lactate Assay Kit (Sigma) according to the manufacturer's instructions.

Blood collection was performed by sub-mandibular bleeding. Blood was allowed to drip into EDTA-containing polypropylene microtubes (Becton Dickinson). Blood in the tube was immediately mixed well by tapping and inverting tube five times to ensure proper anticoagulation. Samples were kept at room temperature until analysis (within four hours). Whole EDTA blood samples were analyzed in duplicates for Complete Blood Count (CBC) with leukocyte differential and platelet count on a Hemavet 950FS Multi Species Hematology System (Drew Scientific) programmed with mouse hematology settings. For glucose and lactate analysis, blood was allowed to drip into heparin-containing polypropylene microtubes (Becton Dickinson). Blood in the tube was immediately mixed well by tapping and inverting tube five times to ensure proper anticoagulation. To obtain plasma, blood was immediately centrifuged after collection at 3,000 g at 4° C. Plasma glucose concentration was determined using the Glucose (GO) Assay Kit (Sigma) according to the manufacturer's instructions. Plasma lactate concentration was determined using the Lactate Assay Kit (Sigma) according to the manufacturer's instructions.

### Behavioral Analysis.

Behavioral testing was carried out at the Salk Institute for Biological Studies Behavioral Testing Core. Basic sensorimotor function was assessed in the Open Field Test, Rotarod, Grip Strength, and Neurological Screen.

Behavioral testing was carried out at the Salk Institute for Biological Studies Behavioral Testing Core. Basic sensorimotor function was assessed in the Open Field Test, Rotarod, Grip Strength and Neurological Screen. Tests were carried out in the order above and separated by a 24 hr rest period to reduce carry-over effects. Locomotor Activity. The Open Field test measure baseline levels of locomotor activity in freely moving mice. Mice are individually placed into clear Plexiglas boxes (40.6 3 40.6 3 38.1 cm) surrounded by multiple bands of photo beams and optical sensors that measure horizontal (ambulatory) and vertical (rearing) activity (Med Associates, USA). Their movement is detected as breaks within the beam matrices and automatically recorded for 60 min.

Motor Coordination. The Rotarod test measures locomotor coordination on a gradually accelerating spinning rod. Mice are placed on an elevated spinning rod and the latency and speed at which a fall occurs is automatically recorded (San Diego Instruments, USA. The test is conducted in two phases beginning with a 60 s training session at a constant speed of 3 RPM followed by 4 trials using an accelerating ramp profile of 0-30 RPM in 300 s. To examine potential differences in motor learning, the test is repeated 24 hr later.

Neuromuscular Function. The Grip Strength test (GS) measures grip force in the forelimbs. The apparatus consists of an acrylic platform with two horizontally mounted digital gauges connected to each end of the platform (San Diego Instruments, USA). The digital gauges record both compression and tension using the displacement force exerted onto a metal bar attached to each gauge. When determining forelimb strength, the animal is lowered toward the metal bar until it grips the bar with its forepaws and is gently tugged away in a horizontal motion. The maximum tension force is recorded upon release. This procedure is repeated 4 times and the average force in grams is calculated for each animal.

Neurological Screen. Gross sensory function is assessed using a subset of tests from the primary SHIRPA protocol (Rogers et al., 1997). This assessment includes screening the presence or absence of the righting response, corneal reflex (eye blink response to direct air puff stimulus), pinna reflex (ear twitch), visual acuity (pupillary light reflex and reaching), olfactory response to aversive stimuli, and the startle response.

### Array Comparative Genomic Hybridization.

aCGH was performed following Agilent Oligonucleotide Array-Based CGH for Genomic DNA Analysis (Agilent Technologies, Santa Clara, Calif.).

aCGH was performed following Agilent Oligonucleotide Array-Based CGH for Genomic DNA Analysis (Agilent Technologies, Santa Clara, Calif.) protocol version 7.3. Labeled DNA was hybridized to the 4×180k SurePrint G3 Mouse CGH Microarray with the design based on NCBI37/mm9 and an overall median probe spacing of 10.9 kb (9.1 kb in Refseq genes). The microarray was scanned with the Agilent C Scanner (Agilent Technologies). Normalization was performed with Agilent Feature Extraction software and analyzed with Agilent Genomic Workbench (Agilent Technologies).

### Exome Capture and High-Throughput Sequencing.

Exome capture was using the SeqCap EZ Mouse Exome Design probe pool (54 Mb, NimbleGen) according to the manufacturer's protocol. Exome capture was performed on pooled libraries generated from tail DNA of one animal of each inbred parental strain and from two male and two female mito-ApaLI offspring, using the SeqCap EZ Mouse Exome Design probe pool (54 Mb, NimbleGen) according to the manufacturer's protocol. Capture libraries were sequenced for 84 cycles on an Illumina HiSeq 2500. Exome coverage was 18× and 70× for the parentals and 61× to 92× for the offspring. Sequencing reads were aligned to the mm9 reference genome using bwa, then potential variants were detected using samtools, considering only sites where C1 and C4 matched the reference sequence and genotype quality met a Q30 threshold.

### Statistical Evaluation.

Statistical analyses were performed by using standard unpaired Student's t test with Welch's correction using Prism 6 software (GraphPad). All data are presented as mean±SEM and represent a minimum of two independent experiments. Statistical significance is displayed as *p<0.05, **p<0.01, ***p<0.001, and ****p<0.0001.

### Accession Numbers.

The GEO database accession number for the aCGH data sets reported in this paper is GSE67371. The GEO accession number for the exome sequencing data sets reported in this paper is SRP056327.

### REFERENCES

Alexeyev, M. F., Venediktova, N., Pastukh, V., Shokolenko, I., Bonilla, G., and Wilson, G. L. (2008). Selective elimination of mutant mitochondrial genomes as therapeutic strategy for the treatment of NARP and MILS syndromes. Gene Ther. 15, 516-523.
Alexeyev, M., Shokolenko, I., Wilson, G., and LeDoux, S. (2013). The maintenance of mitochondrial DNA integrity—critical analysis and update. Cold Spring Harb. Perspect. Biol. 5, a012641-a012641.
Anderson, S., Bankier, A. T., Barrell, B. G., de Bruijn, M. H., Coulson, A. R., Drouin, J., Eperon, I. C., Nierlich, D. P., Roe, B. A., Sanger, F., et al. (1981). Sequence and organization of the human mitochondrial genome. Nature 290, 457-465.
Bacman, S. R., Williams, S. L., Garcia, S., and Moraes, C. T. (2010). Organ-specific shifts in mtDNA heteroplasmy following systemic delivery of a mitochondria-targeted restriction endonuclease. Gene Ther. 17, 713-720.
Bacman, S. R., Williams, S. L., Duan, D., and Moraes, C. T. (2012). Manipulation of mtDNA heteroplasmy in all striated muscles of newborn mice by AAV9-mediated delivery of a mitochondria-targeted restriction endonuclease. Gene Ther. 19, 1101-1106.
Bacman, S. R., Williams, S. L., Pinto, M., Peralta, S., and Moraes, C. T. (2013). Specific elimination of mutant mitochondrial genomes in patient-derived cells by mitoTALENs. Nat. Med. 19, 1111-1113.
Brown, D. T., Herbert, M., Lamb, V. K., Chinnery, P. F., Taylor, R. W., Lightowlers, R. N., Craven, L., Cree, L., Gardner, J. L., and Turnbull, D. M. (2006). Transmission of mitochondrial DNA disorders: possibilities for the future. Lancet 368, 87-89.
Chinnery, P. F., Thorburn, D. R., Samuels, D. C., White, S. L., Dahl, H. M., Turnbull, D. M., Lightowlers, R. N., and Howell, N. (2000). The inheritance of mitochondrial DNA heteroplasmy: random drift, selection or both? Trends Genet. 16, 500-505.
Christian, M., Cermak, T., Doyle, E. L., Schmidt, C., Zhang, F., Hummel, A., Bogdanove, A. J., and Voytas, D. F. (2010). Targeting DNA double-strand breaks with TAL effector nucleases. Genetics 186, 757-761.
Craven, L., Tuppen, H. A., Greggains, G. D., Harbottle, S. J., Murphy, J. L., Cree, L. M., Murdoch, A. P., Chinnery, P. F., Taylor, R. W., Lightowlers, R. N., et al. (2010). Pronuclear transfer in human embryos to prevent transmission of mitochondrial DNA disease. Nature 465, 82-85.
Cree, L. M., Samuels, D. C., and Chinnery, P. F. (2009). The inheritance of pathogenic mitochondrial DNA mutations. Biochim. Biophys. Acta 1792, 1097-1102.
Dyall, S. D., Brown, M. T., and Johnson, P. J. (2004). Ancient invasions: from endosymbionts to organelles. Science 304, 253-257.
Gammage, P. A., Rorbach, J., Vincent, A. I., Rebar, E. J., and Minczuk, M. (2014). Mitochondrially targeted ZFNs for selective degradation of pathogenic mitochondrial genomes bearing large-scale deletions or point mutations. EMBO Mol. Med. 6, 458-466.
Haas, R. H., Parikh, S., Falk, M. J., Saneto, R. P., Wolf, N. I., Darin, N., and Cohen, B. H. (2007). Mitochondrial disease: a practical approach for primary care physicians. Pediatrics 120, 1326-1333.
Hayden, E. C. (2013). Regulators weigh benefits of ‘three-parent’ fertilization. Nature 502, 284-285.
Jenuth, J. P., Peterson, A. C., Fu, K., and Shoubridge, E. A. (1996). Random genetic drift in the female germline explains the rapid segregation of mammalian mitochondrial DNA. Nat. Genet. 14, 146-151.
Jun, A. S., Brown, M. D., and Wallace, D. C. (1994). A mitochondrial DNA mutation at nucleotide pair 14459 of the NADH dehydrogenase subunit 6 gene associated with maternally inherited Leber hereditary optic neuropathy and dystonia. Proc. Natl. Acad. Sci. USA 91, 6206-6210.
Marc, P., Margeot, A., Devaux, F., Blugeon, C., Corral-Debrinski, M., and Jacq, C. (2002). Genome-wide analysis of mRNAs targeted to yeast mitochondria. EMBO Rep. 3, 159-164.
Minczuk, M., Papworth, M. A., Kolasinska, P., Murphy, M. P., and Klug, A. (2006). Sequence-specific modification of mitochondrial DNA using a chimeric zinc finger methylase. Proc. Natl. Acad. Sci. USA 103, 19689-19694.
Minczuk, M., Papworth, M. A., Miller, J. C., Murphy, M. P., and Klug, A. (2008). Development of a single-chain, quasi-dimeric zinc-finger nuclease for the selective degradation of mutated human mitochondrial DNA. Nucleic Acids Res. 36, 3926-3938.
Paull, D., Emmanuele, V., Weiss, K. A., Treff, N., Stewart, L., Hua, H., Zimmer, M., Kahler, D. J., Goland, R. S., Noggle, S. A., et al. (2013). Nuclear genome transfer in human oocytes eliminates mitochondrial DNA variants. Nature 493, 632-637.
Reinhardt, K., Dowling, D. K., and Morrow, E. H. (2013). Medicine. Mitochondrial replacement, evolution, and the clinic. Science 341, 1345-1346.
Rogers, D. C., Fisher, E. M., Brown, S. D., Peters, J., Hunter, A. J., and Martin, J. E. (1997). Behavioral and functional analysis of mouse phenotype: SHIRPA, a proposed protocol for comprehensive phenotype assessment. Mamm. Genome 8, 711-713.
Ross, J. M., Stewart, J. B., Hagstrom, E., Brene, S., Mourier, A., Coppotelli, G., Freyer, C., Lagouge, M., Hoffer, B. J., Olson, L., and Larsson, N. G. (2013). Germline mitochondrial DNA mutations aggravate ageing and can impair brain development. Nature 501, 412-415.
Russell, O., and Turnbull, D. (2014). Mitochondrial DNA disease-molecular insights and potential routes to a cure. Exp. Cell Res. 325, 38-43.
Sanjana, N. E., Cong, L., Zhou, Y., Cunniff, M. M., Feng, G., and Zhang, F. (2012). A transcription activator-like effector toolbox for genome engineering. Nat. Protoc. 7, 171-192.
Shoubridge, E. A., and Wai, T. (2007). Mitochondrial DNA and the mammalian oocyte. Curr. Top. Dev. Biol. 77, 87-111.
Tachibana, M., Amato, P., Sparman, M., Woodward, J., Sanchis, D. M., Ma, H., Gutierrez, N. M., Tippner-Hedges, R., Kang, E., Lee, H.-S., et al. (2013). Towards germline gene therapy of inherited mitochondrial diseases. Nature 493, 627-631.
Taylor, R. W., and Turnbull, D. M. (2005). Mitochondrial DNA mutations in human disease. Nat. Rev. Genet. 6, 389-402.
Vogel, G. (2014). Assisted reproduction. FDA considers trials of ‘three-parent embryos’. Science 343, 827-828.
Wai, T., Ao, A., Zhang, X., Cyr, D., Dufort, D., and Shoubridge, E. A. (2010). The role of mitochondrial DNA copy number in mammalian fertility. Biol. Reprod. 83, 52-62.
Wallace, D. C., and Chalkia, D. (2013). Mitochondrial DNA genetics and the heteroplasmy conundrum in evolution and disease. Cold Spring Harb. Perspect. Biol. 5, a021220.
Wang, T., Sha, H., Ji, D., Zhang, H. L., Chen, D., Cao, Y., and Zhu, J. (2014). Polar body genome transfer for preventing the transmission of inherited mitochondrial diseases. Cell 157, 1591-1604.

What is claimed is:

1. A method of treating a mitochondrial-associated disease, disorder, or condition comprising introducing a composition, which has a mitochondria-targeted nuclease or a nucleic acid construct encoding the mitochondria-targeted nuclease, into a female gametocyte.

2. The method of claim 1, further comprising:
introducing a composition that has the nucleic acid construct encoding the mitochondria-targeted nuclease into the female gametocyte such that the mitochondria-targeted nuclease is expressed in the female gametocyte.

3. The method of claim 1, wherein the mitochondria targeted nuclease targets a mutation in mtDNA associated with the mitochondrial-associated disease, disorder, or condition, the mitochondria-targeted nuclease being present in an amount sufficient to reduce or eliminate mutant mitochondrial DNA (mtDNA) in the female gametocyte.

4. The method of claim 1, wherein the mitochondria targeted nuclease is introduced in an amount sufficient to prevent the transgenerational transmission of mutant mtDNA associated with the mitochondrial-associated disease, disorder, or condition.

5. The method of claim 1, further comprising:
fertilizing the female gametocyte after the introducing of the mitochondria targeted nuclease that eliminated the mitochondrial-associated disease, disorder, or condition.

6. The method of claim 1, wherein the mitochondria targeted nuclease comprises a restriction enzyme selected from the group consisting of mito-ApaLI, Xmal, mito-TALEN, ZFN, HindIII, BcII, and BsrI.

7. The method of claim 1, wherein the mitochondria targeted nuclease comprises a mitochondria-targeted transcription activator-like effector nuclease (TALEN).

8. The method of claim 1, wherein the mitochondrial associated disease disorder, or condition is selected from the group consisting of:
Neuropathy ataxia retinitis pigmentosa (NARP) syndrome; mitochondrial encephalo-myopathy with lactic acidosis and stroke like episodes (MELAS); maternally inherited Leigh's syndrome (MILS) syndrome; NARP-MILS syndrome; hemolytic anemia; a neurodegenerative disease; diabetes; mitochondrial dysfunction-associated aging; gastro-intestinal disorders; cardiac disease; liver disease; diabetes; respiratory disease; seizures; visual/hearing loss; lactic acidosis; developmental delays; mitochondrial myopathy; diabetes mellitus; diabetes mellitus and deafness (DAD); Leber's hereditary optic neuropathy (LHON); Leber's hereditary optic neuropathy and dystonia (LHOND); Wolff-Parkinson-White syndrome; multiple sclerosis-type disease; Leigh syndrome; subacute sclerosing encephalopathy; seizures; altered states of consciousness; dementia; ventilatory failure; neuropathy, ataxia, retinitis pigmentosa, and ptosis (NARP); myoneurogenic gastrointestinal encephalopathy (MNGIE); gastrointestinal pseudo-obstruction; neuropathy; Myoclonic Epilepsy with Ragged Red Fibers (MERRF); progressive myoclonic epilepsy; hearing loss; Mitochondrial myopathy; encephalomyopathy; lactic acidosis; mtDNA depletion; mitochondrial neurogastrointestinal encephalomyopathy (MNGIE); and Friedreich's ataxia

9. The method of claim 1, further comprising:
performing pre-implantation genetic diagnosis (PGD) on the female gametocyte or an embryo formed from the female gametocyte; and selecting a female gametocyte or an embryo with a sufficient mitochondrial DNA load based on the results of PGD, wherein the sufficient mitochondrial DNA load is an amount sufficient for an embryo to successfully implant into a uterus.

10. The method of claim 1, further comprising:
implanting the female gametocyte after introduction of the mitochondria targeted nuclease into a female uterus; or implanting a fertilized female gametocyte after introduction of the mitochondria-targeted nuclease into a female uterus.

11. The method of claim 1, wherein the introducing includes implementing a transfection process that targets a mutated mitochondrial genome by:
transfecting a single cell having mutated mitochondrial deoxyribonucleic acid (mtDNA) with the mitochondria-targeted nuclease or the nucleic acid construct encoding the mitochondria-targeted nuclease of the composition to effect induction of mtDNA heteroplasmy shift to destroy a sufficient quantity of mutated mtDNA so as to lessen a percentage of mutated mtDNA in the single cell with respect to total mtDNA so as to yield a percentage of mutated mDNA remaining that is below a threshold of 60 to 90 percent so that the single cell does not exhibit a diseased state from the mutated mtDNA, the single cell being selected from the group consisting of an oocyte and a zygote; wherein the transfecting is effected with the mitochondria targeted nuclease or the nucleic acid construct encoding the mitochondria-targeted nuclease of the composition in a sufficiently purified form that targets just the mutated mtDNA for destruction without impairing funcundity of the single cell after subsequent mitosis of the single cell.

12. The transfection process of claim 11, wherein the mitochondria targeted nuclease or the nucleic acid construct encoding the mitochondria-targeted nuclease of the composition is selected from the group consisting of a restriction endonuclease and a transcription activator-like effector nuclease (TALENs).

13. The transfection process of claim 11, wherein the transfecting is carried out by transfecting the single cell with a messenger ribonucleic acid (mRNA) having the mitochondria targeted nuclease.

14. The transfection process of claim 11, further comprising the step of:
synthesizing the mitochondria targeted nuclease or the nucleic acid construct encoding the mitochondria-targeted nuclease of the composition in a sufficient quantity and purity to destroy a sufficient amount of the mtDNA to lower the percentage of the mutated mtDNA remaining to below the threshold.

15. The transfection process of claim 11, wherein the synthesizing includes performing in vitro transcription of messenger ribonucleic acid (mRNA) with a linearized and gel purified (Qiagen) plasmid template and then purifying and quantifying the mRNA, the transfecting being carried out by transfecting the single cell with the mRNA, which has the mitochondria targeted nuclease or the nucleic acid construct encoding the mitochondria-targeted nuclease of the composition.

16. An apparatus for administering treatment for a mitochondrial-associated disease, disorder, or condition comprising:
means for introducing a composition, which has a mitochondria-targeted nuclease or a nucleic acid construct encoding the mitochondria-targeted nuclease, into a female gametocyte.

17. The apparatus of claim 16, wherein said means for introducing includes means for effecting a transfection process that targets a mutated mitochondrial genome by transfecting a single cell having mutated mitochondrial deoxyribonucleic acid (mtDNA) with mitochondria targeted nuclease or the nucleic acid construct encoding the mitochondria-targeted nuclease of the composition to effect the induction of the mtDNA heteroplasmy shift to destroy a sufficient quantity of the mutated mtDNA so as to lessen a percentage of mutated mtDNA in the single cell with respect to total mtDNA so as to yield a percentage of mutated mDNA remaining that is below a threshold of 60 to 90 percent so that the single cell does not exhibit a diseased state from the mutated mtDNA, the single cell being selected from the group consisting of an oocyte and a zygote, wherein the transfecting is effected with the mitochondria targeted nuclease or the nucleic acid construct encoding the mitochondria-targeted nuclease of the composition in a sufficiently purified form that targets just the mutated mtDNA for destruction without impairing funcundity of the single cell after subsequent mitosis of the single cell.

18. The apparatus of claim 16, wherein the mitochondria targeted nuclease is selected from the group consisting of restriction endonuclease and transcription activator-like effector nuclease (TALEN).

19. The apparatus of claim 17, wherein the means for effecting the transfection process is configured to transfect the single cell with a messenger ribonucleic acid (mRNA) having the synthesized mitochondria targeted nuclease or the nucleic acid construct encoding the mitochondria-targeted nuclease of the composition that is encoding mitochondria targeted ApaLI restriction enzyme.

20. The apparatus of claim 17, wherein the means for effecting the transfection process synthesizes the mitochondria targeted nuclease or the nucleic acid construct encoding the mitochondria-targeted nuclease of the composition in a sufficient quantity and purity to destroy a sufficient amount of the mtDNA to lower the percentage of the mutated mtDNA remaining to below the threshold.
